(19) United States
(12) Patent Application Publication
(10) Pub. No.: US 2022/0251007 A1
(43) Pub. Date: Aug. 11, 2022

(54) Industrial Process for Manufacturing of Perfluoropentane (PFP)

(71) Applicant: Fujian Yongjing Technology Co.,Ltd, Shaowu (CN)

(72) Inventors: WEIFEN LUO, Shaowu (CN); RONGWEN DING, Shaowu (CN)

(73) Assignee: Fujian Yongjing Technology Co.,Ltd, Shaowu (CN)

(21) Appl. No.: 17/565,497

(22) Filed: Dec. 30, 2021

Related U.S. Application Data
(63) Continuation of application No. PCT/CN2021/121516, filed on Sep. 29, 2021.

(30) Foreign Application Priority Data
Feb. 1, 2021 (DE) 102021102265.7

(51) Int. Cl. C07C 17/07; C07D 307/33; C07C 17/383

(52) U.S. Cl. CPC C07C 17/07; C07C 17/383; C07D 307/33

(57) ABSTRACT

The invention relates to a new industrial process for manufacturing of perfluoropen-tane (PFP), and to the manufacture of a novel intermediate compound thereof, as well as to the novel intermediate compound itself and the use thereof in the process for manu-facturing of perfluoropentane (PFP). Accordingly, the invention relates to a process for the manufacture of the compound PFP (perfluoropentane), and/or of the compound perfluorinated 4-methylbutyrolactone, i.e., the precursor or intermediate compound of PFP (perfluoropentane), characterized in that the process comprises direct fluorination reaction with F2 gas as the fluorination agent, and/or from the fluorination reaction with SF4 as the fluorination agent. The present invention provides an efficient and simplified new industrial process for manufacturing of perfluoropentane (PFP) and/or of the compound perfluorinated 4-methylbutyrolactone, and preferably enabling large-scale and/or industrial production of perfluoropentane (PFP) and/or of the compound perfluorinated 4-methylbutyrolactone by means of special equipment and special reactor design.

### BACKGROUND OF THE INVENTION

### 1. Field of the Invention

The invention relates to a new industrial process for manufacturing of perfluoropentane (PFP), and to the manufacture of a novel intermediate compound thereof, as well as to the novel intermediate compound itself and the use thereof in the process for manufacturing of perfluoropentane (PFP).

### 2. Description of the Prior Art

Perfluoropentane(PFP) or dodecafluoropentane (UPAC name) is also known under its INN/USAN name as perflenapent.

International nonproprietary name (INN) is an official generic and non-proprietary name given to a pharmaceutical drug or an active ingredient. United States Adopted Names (USAN) are unique nonproprietary names assigned to pharmaceuticals marketed in the United States. Each name is assigned by the USAN Council, which is co-sponsored by the American Medical Association (AMA), the United States Pharmacopeial Convention (USP), and the American Pharmacists Association (APhA).

Perfluoropentane (PFP) (CAS number: 678-26-2) is a fluorocarbon (chemical formula C₅F₁₂; molecular mass 288.036 g/mol), the perfluorinated analogue of pentane, and is a liquid that boils at slightly over room temperature (boiling point 28° C.; vapor pressure 83.99 kPa at 25° C.; density 1.63 g/mL for liquid at 25° C.; viscosity 0.652 mPA*s at 25° C.).

Perfluoropentane (PFP) has several biomedical applications including: propellant for pressurized metered dose inhalers; gas core in microbubble ultrasound contrast agents; and occlusion therapy via the conversion of nanometer liquid droplets into micrometer sized gas microbubbles (acoustic droplet vaporization). In particular, perfluoropentane (PFP) is also used as blood substitute.

A blood substitute, also called artificial blood or blood surrogate, is a substance used to mimic and fulfil some functions of biological blood. It aims to provide an alternative to blood transfusion, which is transferring blood or blood-based products from one person into another. Presently, there are widely available non-blood volume expanders for cases where only volume restoration is required.

The properties of perfluorinated compounds and especially perfluorinated alkanes used as artificial blood substitutes are known since long time as they dissolve oxygen (O₂) very well; for example, as published by G. Motta et al. in Minerva Med. 1983 Jan. 14; 74(1-2):1-18 or N. Shnoy in Anaesthesist. 1979; 28(11), 503-10; and by D. D. Lawson et al. in J. Fluorine Chemistry Volume 12, Issue 3 (1978), 221-236 (https://doi.org/10.1016/S0022-1139(00)81587-0) (“Methods for the estimation of vapor pressures and oxygen solubilities of fluorochemicals for possible application in artificial blood formulations.”).

Experiments where the lungs of a species were filled completely with in perfluorocarbon dissolved oxygen are well known (see, for example, Shaffer, Thomas H.; Wolfson, Marla R.; Clark, Leland C. (October 1992), “Liquid ventilation”, Pediatric Pulmonology. 14 (2): 102-109.doi:10.1002/ppul.1950140208). A more recent review was published by S. Sarkar in Indian J Crit Care Med. 2008 July-September; 12(3): 140-144 and can be found in https://www.ijccm.org/doi/pdf/10.4103/0972-5229.43685.

Especially perfluoropentane has excellent properties in dissolving oxygen (O₂) and also usage as pharmaceutical surfactant, for example, as published by M. A. Kandatai et al. in Langmuir. 2010 Apr. 6; 26(7): 4655-4660, as contrast media as published by Sonus Pharmaceutical in U.S. Pat. No. 5,558,854 (1996).

Due to the global pandemic Corona virus in 2020, artificial blood gains renewed huge attendance to treat patients with severe length defects and breathing problems.

Unfortunately synthesis and final purification of perfluorinated compounds to an acceptable quality for use in humans in general is very challenging, because partially fluorinated compounds are very toxic, and sometimes use in humans is even impossible due to uncomplete fluorination reaction and/or due to a too low selectivity of chemical reaction, or just due to the use of impure raw materials.

Hence, purity is a key parameter for pharmaceutical applications in human beings, and sometimes more efforts need to be spent for the purification (of raw materials, intermediates and/or products) rather than for the synthesis itself.

The existing main technologies for making perfluorinated compounds are either by so-called telomerization reaction (a linear dimerization of 1,3-dienes with simultaneous addition of a nucleophile in a catalytic reaction, the C—F-bond is formed already) which mostly always leads to mixtures of compounds with different chain lengths, or by electrofluorination (electrochemical fluorination, with local in situ generation of F₂ at electrodes) in HF as solvent, and disadvantages such as, for example, raw material which is expensive due to electric power consumption, often low selectivity due to uncomplete fluorination and/or C—C-bond breaking. Furthermore, on the one side, achievable yields in electrofluorination are quite low due to the needed purification, and achievable conversions are low as often organic material deposes on electrodes and thereby can cause stopping the reaction. Also, the electrofluorination only is done in batch.

Perfluoropentane (PFP) is a leading candidate for usage as artificial blood, as propellant, as heat transfer fluid and for usages in metered dose inhalers in asthma sprays.

A known preparation method for perfluoropentane (PFP) is a two-step process by telomerization of hexafluoropropylene (HFP) and tetrafluoroethylene (TFE) followed by addition of fluorine (F₂) to the double bond, for example, as disclosed by Asahi Glass in EP967191 (1999) according to:

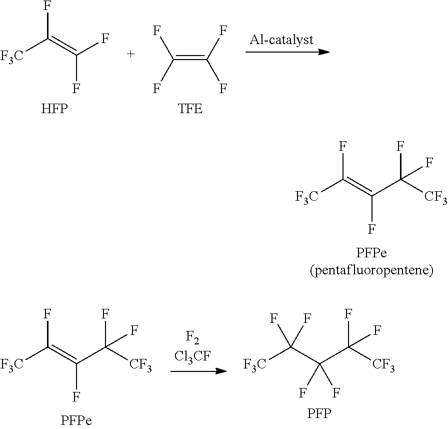

The reaction of hexafluoropropylene (HFP) with tetrafluoroethylene (TFE) was also already described, for example, in Du Pont's patent U.S. Pat. No. 5,268,122 (1993), in Example C in good yield and as perfluoro-2-pentene (PFPe) cis/trans isomer intermediate in a ratio of 11:89, but no purity and lifetime of the Al-catalyst (prepared by AlCl₃+HF) is given; a onetime usage of the Al-catalyst can be estimated as Alumina based catalysts are known to absorb “F-atoms” and to become deactivated. Besides this technical drawback of lifetime of the telomerization catalyst, achievable PFP purity in industrial scale is another drawback of this sequence. Of course TFE and HFP are known to undergo polymerisations with themselves once activated especially in presence of Lewis acids like AlClₓF_{y}. The fluorination of pentane by using CoF₃ as fluorinating agent was already described by Burford et al., Ind. Eng. Chem., 39, 1947, 328 but besides missing achievable purity after purification with this reaction industrial scalability is not given as much aqueous fluoride containing very toxic and corrosive waste will be formed.

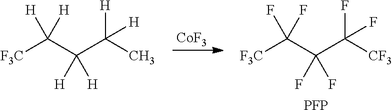

Also the preparation of polyfluoralkanes with higher concentrated F₂ (fluorination of mixtures) is described by Solvay Fluor GmbH in EP0619287 (1994) and especially of PFP with F₂ out of Pentane by Allied Signal in EP0031519 (1981), example 3 in a Fused Alumina Porous Tube. In Progress in Inorganic Chemistry 1979, 161 ff, Richard J. Lagow and John L. Margrave disclose many types of compounds (but not PFP) made with fluorination with F₂-gas, but there is NO suitable reactor type for industrial scale. In U.S. Pat. No. 5,093,432 (1992) some reactor types (101 scale) are disclosed but as this are just “vessels” and no “tubes”, contact time between F₂-gas and the liquid with the substrate as well as heat exchange is very bad and led to very long reaction times and loss of some F₂— gas especially in reactor types like shown in FIG. 1, a little bit better in FIG. 2 using a recirculating stream but all this which results in bad space/time yields and finally does not fit for industrial scale production. No material of construction is mentioned and this is another key parameter for a successful fluorination with F₂. All this known processes and reactor types published in that journals and patents mentioned above do not fit either regarding availability of raw material, selectivity, achievable purity for Pharma application or just scalability to economic industrial scale as no suitable reactor type is disclosed.

The existing demand in the field of an efficient process, e.g., also industrially applicable process, for manufacturing of perfluoropentane (PFP) is solved by the present invention.

All existing methods in the state of the art to prepare perfluoropentane (PFP) involve several challenges which are, for example:

All these challenges lead to quite high manufacturing costs, high consumption of energy and much toxic waste formation, e.g., formation of undesired salts and/or undesired organic compounds.

As shown herein before the prior art processes are not yet optimal and have several disadvantages.

Accordingly, there is a high demand of enabling large-scale and/or industrial production of perfluoropentane (PFP), and as perfluoropentane (PFP) is an aliphatic organic compound with five C-atoms, especially starting from easily and in high purity accessible (cyclo)aliphatic starting material compound having a total of five C-atoms.

Thus, it is an object of the present invention to provide an efficient and simplified new industrial process for manufacturing of perfluoropentane (PFP), and/or of the compound perfluorinated 4-methylbutyrolactone, respectively, which may serve as an intermediate compound in the manufacture of said perfluoropentane (PFP).

It is a further object of the present invention to also provide an efficient and simplified new industrial process for manufacturing of perfluoropentane (PFP) via or out of the compound perfluorinated 4-methylbutyrolactone.

It is preferably another object of the present invention to provide an efficient and simplified new industrial process for manufacturing of perfluoropentane (PFP) and/or of the compound perfluorinated 4-methylbutyrolactone, and preferably enabling large-scale and/or industrial production of perfluoropentane (PFP) and/or of the compound perfluorinated 4-methylbutyrolactone by means of special equipment and special reactor design.

### SUMMARY OF THE INVENTION

The object of the invention is solved as defined in the claims, and described herein after in detail. The invention relates to a new industrial process for manufacturing of perfluoropentane (PFP).

FIG. 1 shows the manufacture of PFP (perfluoropentane), both reaction steps (one step with F2 gas as fluorination agent, e.g., as the first step.; one step with SF₄ as fluorination agent, e.g., as the second step), using a counter current reactor system (e.g., a gas scrubber system). See Example 2. Starting material compound is 4-methyl-butyrolactone; intermediate compound is perfluorinated 4-methyl-butyrolactone. The intermediate perfluorinated 4-methyl-butyrolactone compound can be isolated, if desired, and in that case only the first step with and F₂-gas as fluorination agent is performed. The isolated perfluorinated 4-methyl-butyrolactone compound can be used as starting material compound, if desired, for the manufacture of the PFP (perfluoropentane) by subjecting the perfluorinated 4-methyl-butyrolactone compound to a fluorination reaction with SF₄ as fluorination agent. During the fluorination reaction with SF₄ as fluorination agent, also the lactone ring opening takes place.

FIG. 2 shows the manufacture of PFP (perfluoropentane) in a microreactor system (two microreactors) in continuous manner; both reaction steps (one step with F₂-gas as fluorination agent, e.g., as the first step; one step with SF₄ as fluorination agent, e.g., as the second step). See Example 3. Starting material compound is 4-methyl-butyrolactone; intermediate compound is perfluorinated 4-methyl-butyrolactone. The intermediate perfluorinated 4-methyl-butyrolactone compound can be isolated, if desired, and in that case only the first step with and F₂-gas as fluorination agent is performed. The isolated perfluorinated 4-methyl-butyrolactone compound can be used as starting material compound, if desired, for the manufacture of the PFP (perfluoropentane) by subjecting the perfluorinated 4-methyl-butyrolactone compound to a fluorination reaction with SF₄ as fluorination agent. During the fluorination reaction with SF₄ as fluorination agent, also the lactone ring opening takes place.

FIG. 3 shows the manufacture of PFP (perfluoropentane) in a microreactor system, wherein the starting material compound is HFAA (hexafluoro acetyleacetone, with two microreactors in continuous manner; both reaction steps (one step with SF₄ as fluorination agent, e.g., as the first step; one step with F₂-gas as fluorination agent, e.g., as the second step). See Example 4. The intermediate compound, decafluoro-3,3-dihydro-pentane, can be isolated, if desired, and in that case only the first step with and F₂-gas as fluorination agent is performed. The isolated decafluoro-3,3-dihydro-pentane compound can be used as starting material compound, if desired, for the manufacture of the PFP (perfluoropentane) by subjecting the decafluoro-3,3-dihydro-pentane compound to a direct fluorination reaction with F₂-gas as fluorination agent.

Surprisingly, now it has been found that an (cyclo)aliphatic starting material compound having a total of five C-atoms, and wherein two of said C-atoms are part of a carbonyl-group (—C(═O)—) function, can easily be perfluorinated in a two-step process to perfluoropentane (PFP), which is also known as dodecafluoropentane (UPAC name) or under its INN/USAN name as perflenapent, and wherein in one (fluorination) process step the two said C-atoms being part of said carbonyl-group (—C(═O)—) function are converted into two (—CF₂-)-groups.

The said (cyclo)aliphatic starting material compound having a total of five C-atoms, and wherein two of said C-atoms are part of said carbonyl-group (—C(═O)—) function, can either be a 1,4-lactone compound or a 2,4-diketone compound (also named β-diketone compound), or in other words, the said carbonyl-group (—C(═O)—) function, wherein two of said C-atoms are part thereof in the (cyclo)aliphatic starting material compound having a total of five C-atoms, can either be a 1,4-lactone function or a2,4-diketone function (also named β-diketone function).

Lactones are cyclic carboxylic esters, containing a 1-oxacycloalkan-2-one structure (—C(═O)—O—), which is acyclic carbonyl-group (—C(═O)—) function inthe context of the present invention, involving two C-atoms when forming the lactone cycle, and, for example, the compound 4-methylbutyrolactone is the preferred cycloaliphatic starting material compound. Lactones are formed by intramolecular esterification of the corresponding hydroxycarboxylic acids, which takes place spontaneously when the ring that is formed is five membered, as in the present invention.

A 2,4-diketone (function) or β-diketone (function) is a dicarbonyl compound, i.e., a compound containing two carbonyl (C═O) groups in 2,4-positions, which together are an aliphatic (—C(═O)—) function in the context of the present invention, in the aliphatic starting material compound having a total of five C-atoms. The properties of a 2,4-diketone function or β-diketone function often differ from those of monocarbonyls, and so they are usually considered functional groups of their own. Preferred 2,4-diketones or β-diketones as aliphatic starting material compounds in the context of the present invention are, for example, are acetylacetone, 1,1,1-trifluoro-2,4-pentanedione (trifluoro acetylacetone; TFAA) and hexafluoro acetylacetone (HFAA).

Thus, the present invention describes a new industrial process starting from raw materials, i.e., a (cyclo)aliphatic starting material compound having a total of five C-atoms, wherein a“some acidity” generating group in form of said two C-atoms which are part of said carbonyl-group (—C(═O)—) function, are present—this provides higher activity—allows for softer reaction parameters—and results in higher raw material yield and purity; these starting materials according to the invention are β-diketones or lactones like 4-methylbutyrolactone.

The term “some acidity generating group” is to be understood as an electron-withdrawing group which, in the alpha position to the “some acidity generating group”. Such a group facilitates the splitting off of a hydrogen atom and its substitution. Examples of such “some acidity generating group” are said 1,4-lactone function or a 2,4-diketone function (also named β-diketone function).

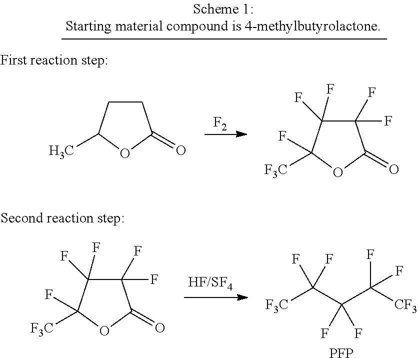

For example 4-methylbutyrolactone can be fluorinated in batch in a STR system (Stirred Tank Reactor system) with F₂-gas (fluorination gas) diluted by inert gas or continuously with diluted by inert gas or concentrated F₂-gas (diluted or concentrated fluorination gas) under anhydrous conditions in a counter-current system or microreactor (preferably out of SiC) to give perfluorinated 4-methylbutyrolactone which as a new compound hitherto unknown. In particular, high concentrations of F₂-gas (fluorination gas) can only be applied in a counter-current reactor system or in microreactor- and coil reactor system, in order to avoiding hot spots which is not possible in a STR system.

Preferably, the said batch (direct) fluorination reaction with F₂-gas (fluorination gas) diluted by inert gas or continuous (direct) fluorination reaction with F₂-gas (fluorination gas) diluted by inert gas or with concentrated F₂-gas is performed under anhydrous conditions. If the degree of fluorination is too low (detectable by the presence of H-atoms measured by NMR) a post fluorination step can be added if needed.

Otherwise and in most trials especially in reactions performed continuously in a microreactor, the yield of perfluorinated 4-methylbutyrolactone is very high and with efficient temperature control almost up to quantitative. If by chance some traces of water might be present in the starting material or in the equipment before use, e.g. from cleaning, some partial position unselective fluorinated gamma-hydroxy butyric acid can be detected. This can be avoided by using properly dried starting material with no moisture content.

No solvent is needed for the first step if done in a microreactor system, preferably made out of SiC (but Hastelloy will work too), alternatively some perfluoropentane (PFP) as solvent for dilution (and as exothermicity buffer) is advantageous at least during startup of a continuous reaction in microreactor or in general if feed control might not be very constant due to installed pump or feed from a slightly pressurized vessel.

Asahi glass also has used PFP already as solvent in their fluorination step (Example 4, EP967191 (1999). Also anhydrous HF serves as solvent for the first step as it is also equimolar formed during the reaction and does not add the need for an additional purification step.

If the F₂-gas is diluted with inert gas, e.g., out of a pressurized cylinder is used, a very high gas load in a microreactor which is less advantageous due to inert gas bubbles in the channels leads to much lower selectivity so it is preferred not to use much (or very) diluted F₂ but optionally an F₂-gas coming directly out of an electrolysis cell used for F₂ production out of HF. Some (potential) CF₄ in the F₂-gas as impurity (comes from some fluorination of carbon electrodes) does NOT inhibit or disturb and also not accelerate the reaction.

For the second step, SF₄ in HF is the preferred reagent for lactone opening and finalization of the fluorination to perfluoropentane, for acceleration of the lactone opening step optionally Lewis acids like TiClₙF₄₋ₙ, SnClnF₄₋ₙ, SbClₘF₅₋ₘ can be added. Many reactions of SF₄ are described e.g. by G. A. Boswell in Organic Reactions (New York) (1974), 21, 1-124 and Wang, Chia Lin J., Organic Reactions (New York) (1985), 34, 319. Also in Houben Weyl Volume E10a, chapter 8, page 321ff (ISBN: 9783131815446) a summary on SF₄-chemistry with experimental procedures is given but for scientific purpose only and NOT suitable for industrial scale. This inventive 2^{nd} step disclosed here can be done batchwise in a STR or continuously in a series of STR or better in a microreactor or series of microreactors optionally made out of SiC but Hastelloy (preferred Hastelloy C4) works well as material of construction.

Commercial fluorinating agents like DAST (diethylaminosulfur trifluoride), Deoxo-Fluor (bis-(2-methoxyethyl)amino sulfur trifluoride), Xtal-Fluor-E (diethylamino)difluorsulfonium-tetrafluoroborat), Xtal-Fluor-M (difluoro(morpholino)sulfoniumtetrafluoroborate), Fluorlead(4-tert-butyl-2,6-dimethylphenylsulfur trifluoride) and PhenoFluor (1,3-bis(2,6-diisopropylphenyl)-2,2-difluoro-4-imidazoline), see formulas given below, in principle can replace SF₄ as fluorinating agent technology wise but will create lot of aqueous and organic waste as work up is with hydrolysis or even by preparative chromatography to separate the product perfluoropropane as organic phase from the remaining waste coming from fluorinating agent in the aqueous phase. Due to this, fluorinating agents might not meet economic and environmental needs but are included into this invention. For larger industrial scale, SF₄ is the preferred fluorinating agent. Both steps can be done either in STR, coil- and microreactor or countercurrent reactor or a combination of different reactor types. Accordingly, said fluorination agents are not suitable for the process of the invention, but not preferred, as reactions are trickier, and currently these fluorination agents are also very expensive and not available on a large commercial scale.

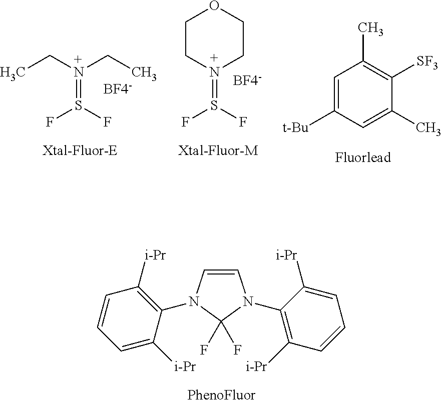

Diethylaminosulfur trifluoride(DAST) is an organo sulfur compound with the formula Et₂NSF₃. The diethylaminosulfur trifluoride is a fluorinating reagent used for the synthesis of organofluorine compounds. The compound is liquid, colourless; older samples assume an orange colour. Upon heating, diethylaminosulfur trifluoride (DAST) converts to the highly explosive (NEt₂)₂SF₂ with expulsion of sulfur tetrafluoride. To minimize accidents, samples are maintained below 50° C.

Bis-(2-methoxyethyl)aminosulfur trifluoride (trade name: Deoxo-Fluor-E) and difluoro(morpholino)sulfonium tetrafluoroborate (trade name: XtalFluor-M) are reagents derived from DAST with less explosive potential.

Sulfur tetrafluoride (SF₄) is the preferred fluorination agent in the process of the invention. Sulfur tetrafluoride (SF₄) is a colorless corrosive gas that releases dangerous HF (hydrogen fluoride) upon exposure to water or moisture. Despite these unwelcome characteristics, this compound is a useful reagent for the preparation of organofluorine compounds, some of which are important in the pharmaceutical and specialty chemical industries.

Besides the synthesis out of a cyclic precursor, the sequence out of commercial available linear precursors like the acetylacetones (includes partial fluorinated acetylacetones) is another option included in this invention but the SF₄ step is done first followed by the final fluorination with F₂ as it is known that Keto group containing substrates sometimes are hardly to fluorinate, an overview is given by Hutchinson et al in Organofluorine Chemistry in the chapter “Elemental Fluorine in Organic Chemistry” 193 (1997), page 1 ff. The reactions are given hereunder:

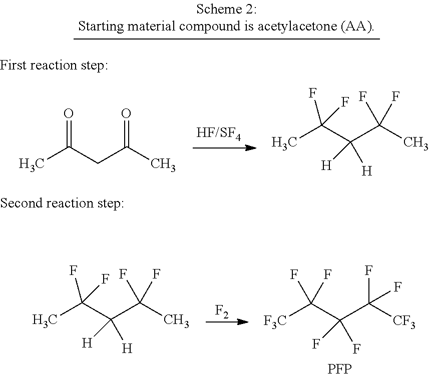

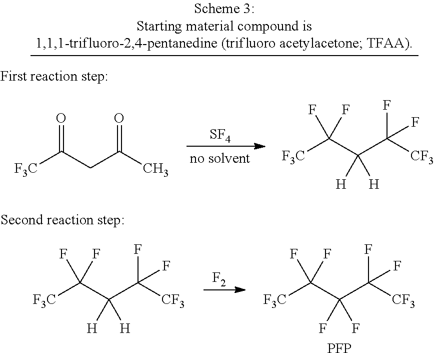

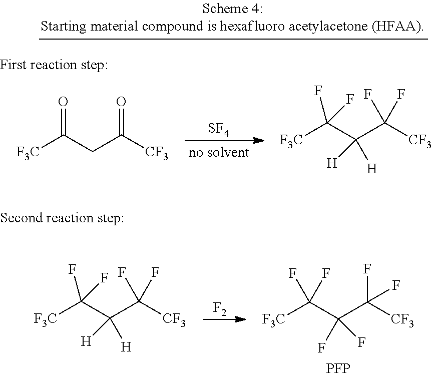

In general the stoichiometric ratio of raw materials vs. F₂ as well as SF₄ (or other fluorinating agents) always has to be adapted to the amount of exchangeable hydrogen atoms respectively to the amount of carbonyl groups which have to be converted to —CF₂-groups. Another big advantage of the inventive sequences in industrial scale is that aqueous work up can be avoided in both steps. Generated HF can be removed by simply adding some vacuum or using some inert gas as trailing gas. All formed SOF₂ also can be collected and further used for fluorinations.

The invention relates also to a new industrial process for manufacturing of perfluoropentane (PFP), and/or of the compound perfluorinated 4-methylbutyrolactone, respectively, which is a suitable intermediate in the manufacture of perfluoropentane (PFP), involving reactions in liquid phase and, for example, performing reactions in a counter-current reactor system, in particular in a loop reactor system, or in a counter-current (loop) system (“inverse gas scrubber system”), as well as in a tube reactor system, a continuous flow reactor system, in a coil reactor system or in a microreactor system, preferably performing reactions in a counter-current reactor system or in a microreactor, respectively, as each described here under and in the claims.

Accordingly, the present invention, as described hereunder in more detail and as defined in the claims, in one aspect relates to a new process for the industrial synthesis of perfluoropentane (PFP) out of the compound perfluorinated 4-methylbutyrolactone as an intermediate compound, or directly starting from the compound 4-methylbutyrolactone. In particular, in a preferred aspect of the invention, the new process for the industrial synthesis of perfluoropentane (PFP) or of perfluorinated 4-methylbutyrolactone includes a direct fluorination step with elemental fluorine (F₂) of the compound 4-methylbutyrolactone used as the initial starting material. Here, in another aspect the invention also relates to a new process for the industrial synthesis the compound perfluorinated 4-methylbutyrolactone as final product compound out of the compound 4-methylbutyrolactone.

Alternatively, according to the present invention, as described hereunder in more detail and as defined in the claims, in one aspect relates to a new process for the industrial synthesis of perfluoropentane (PFP) out of a compound selected from the group of preferred 2,4-diketones or β-diketones as aliphatic starting material compounds, which in the context of the present invention are, for example, are acetylacetone, 1,1,1-trifluoro2,4-pentanedione (trifluoro acetylacetone; TFAA) and hexafluoro acetylacetone (HFAA).

The present invention as one reaction step involves a direct fluorination process with F₂-gas as fluorination agent in the manufacture or preparation of the compound perfluoropentane (PFP), in particular by means of special equipment and special reactor design, for example, as shown in FIG. 1 and FIG. 2 or 3, respectively, and as further described hereunder. The special equipment and special reactor design employed by the invention may comprise one or more packed bed towers, e.g., in the form of a gas scrubber system, or one or more microreactors.

In one aspect of the invention, the process of the direct fluorination reaction with F₂-gas as the fluorination agent, and/or the fluorination reaction with SF₄ as the fluorination agent and (if applicable) involving the lactone ring opening, is carried out in an autoclave, in a (closed) column reactor, a counter-current reactor system, in particular in a loop reactor system, or in a counter-current (loop) system (“inverse gas scrubber system”), in a tube reactor system, a continuous flow reactor system, in a coil reactor system or in a microreactor system, preferably in a counter-current reactor system or in a microreactor.

In another aspect of the invention, the process of the direct fluorination reaction with F₂-gas as the fluorination agent, and/or the fluorination reaction with SF₄ as the fluorination agent, and (if applicable) involving the lactone ring opening, is carried out in a counter-current reactor system which is a (closed) column reactor, preferably wherein the counter-current reactor system is a (closed) column reactor, preferably a packed bed tower, more preferred a packed bed tower in the form of a gas scrubber system.

It has been discovered that despite the exothermic character of the direct fluorination reaction with F₂-gas as fluorination agent, e.g., within a given time period (e.g., less than 10 hours, or even less than 5 hours), the reaction of the invention can be performed as a larger scale reaction with high conversion rates, and without major impurities in the resulting fluorinated product. The fluorinated product can be produced in kilogram scale quantities, e.g., the direct fluorination process of the invention with F₂-gas as fluorination agent can be performed in a large-scale and/or industrial production of a fluorinated inorganic compound or fluorinated organic compound, respectively.

Particular examples of performing the process of the present invention are described in the context of the FIG. 1 (closed column reactor system) and FIG. 2 or 3 (microreactor system).

The direct fluorination process using F₂-gas as fluorination agent and the other fluorination process using SF₄ as fluorination agent can be performed independently and separately of each other.

Alternatively, the direct fluorination process using F₂-gas as fluorination agent and the other fluorination process using SF₄ as fluorination agent can be performed subsequently as a two-step process with or without isolating and/or purifying the intermediate fluorination product compound.

Direct Fluorination:

The term “direct fluorination” means introducing one or more fluorine atoms into a compound by chemically reacting a starting compound, e.g. according to the present invention into a compound with elemental fluorine (F₂) such that one or more fluorine atoms are covalently bound into the said compound, thus replacing one or more hydrogen atoms therein.

Accordingly, the direct fluorination of the present invention provides a high efficient process for the manufacture or for preparation of the compound perfluoropentane (PFP), or for the perfluorinated 4-methyl-butyrolactone compound, which can be isolated, if desired, as final product compound and/or intermediate compound, by direct fluorination of the 4-methyl-butyrolactone compound, using fluorine gas (F₂), herein also termed “F₂-fluorination gas” or “F₂-gas” or “F₂-gas as the fluorination agent”.

The F₂-fluorination gas used in the invention can be of any origin. For example, the present invention can also use a F₂-fluorination gas in the direct fluorination step using fluorine gas (F₂), as it comes directly out (e.g., without further purification) of an F₂-electrolysis reactor (fluorine cells), and optionally is only diluted by an inert gas (or mixture thereof) to a desired fluorine (F₂) concentration. Of course, the fluorine gas (F₂) coming from an F₂-electrolysis reactor (fluorine cells), if desired, can also be subjected to purification before it is used in the direct fluorination step; optionally this purified fluorine gas (F₂) originally derived from an F₂-electrolysis reactor (fluorine cells) is only diluted to some extent by an inert gas (or mixture thereof) to a desired fluorine (F₂) concentration.

Purification of the fluorination gas as it is derived from an F₂-electrolysis reactor (fluorine cell), if desired, optionally is possible, to remove a part or all by-products and traces formed in the F₂-electrolysis reactor (fluorine cell), prior to its use as fluorination gas in the process of the present invention. However, in the process of the present invention such a partial or complete purification is not obligatory, and the fluorination gas can be directly used as it comes out of an F₂-electrolysis reactor (fluorine cell), but if desired, optionally is only diluted by inert gas to a desired fluorine (F₂) concentration. When employing an F₂-fluorination gas derived from an F₂-electrolysis reactor (fluorine cell), purified or unpurified, thus it can be diluted by an inert gas, most preferably by nitrogen (N₂), to the extent desired.

The fluorine (F₂) concentration in the F₂-fluorination gas may vary in wide range, for example, of from about 1% by volume of elemental fluorine (F₂) up to about almost 100% by volume of elemental fluorine (F₂), based on the total F₂-fluorination gas composition as 100% by volume. The term “about almost 100% by volume of elemental fluorine (F₂)” means that for technical reason, e.g., if the elemental fluorine (F₂) is taken from a fluorine cell, technical grade elemental fluorine (F₂) will contain traces of impurities, for example some tetrafluoromethane (CF₄) formed during electrolysis. Hence, the term “about almost 100% by volume of elemental fluorine (F₂)” will be understood by the person skilled in the field, e.g., as up to about 99.9%, up to about 99.8%, up to about 99.7%, up to about 99.6%, up to about 99.5%, or up to about 99%+1% respectively, each by volume of elemental fluorine (F₂).

Typical ranges of lower fluorine (F₂) concentrations in the F₂-fluorination gas, for example, are of from about 1% by volume of elemental fluorine (F₂) up to about 30% by volume of elemental fluorine (F₂), more preferably of from about 5% by volume of elemental fluorine (F₂) up to about 25% by volume of elemental fluorine (F₂), even more preferably of from about 5% by volume of elemental fluorine (F₂) up to about 20% by volume of elemental fluorine (F₂), each range based on the total F₂-fluorination gas composition as 100% by volume. The lower fluorine (F₂) concentrations in the F₂-fluorination gas, for example, can be applied when performing reactions in a counter-current reactor system, in particular in a loop reactor system, or in a counter-current (loop) system (“inverse gas scrubber system”).

Typical ranges of higher fluorine (F₂) concentrations in the F₂-fluorination gas, for example, are of from about 85% by volume of elemental fluorine (F₂) up to about almost 100% (as defined herein above) by volume of elemental fluorine (F₂), preferably of from about 90% by volume of elemental fluorine (F₂) up to about almost 100% (as defined herein above) by volume of elemental fluorine (F₂), based on the total F₂-fluorination gas composition as 100% by volume. The higher fluorine (F₂) concentrations in the F₂-fluorination gas, for example, are preferably applied when performing reactions in a tube reactor system, in a continuous flow reactor system, in a coil reactor system, or in a microreactor system, preferably in a microreactor system. However, the said higher fluorine (F₂) concentration in the F₂-fluorination gas, for example, can also be applied when performing reactions in in a counter-current reactor system, in particular in a loop reactor system, or in a counter-current (loop) system (“inverse gas scrubber system”).

It goes without saying that a skilled person will understand that within any of the above given ranges any intermediate values and intermediate ranges can be selected, too.

The term “vol.-%” as used herein means “% by volume”. Unless otherwise stated, all percentages (%) as used herein denote “vol.-%” or “% by volume”, respectively.

The term “inert gas” means a gas that does not undergo chemical reactions under a set of given conditions. Typical inert gases include any noble gas, which make up a class of chemical elements with similar properties, and under standard conditions, are all odorless, colorless, monatomic gases with very low chemical reactivity, for example, such like the noble gases are helium (He), neon (Ne) and argon (Ar), or inert gases such as Nitrogen (N₂). Preferably, (purified) argon (Ar) and/or nitrogen (N₂) gases are used as inert gases due to their high natural abundance (78.3% N₂, 1% Ar in air) and low relative cost. The more preferred inert gas in the context of the invention is nitrogen (N₂). The use of mixtures of said inert gases is possible, too.

The extent of diluting fluorine (F2) gas in an inert gas or mixture thereof, i.e., the fluorine (F₂) concentration of the F₂-fluorination gas used in the fluorination process step, can depend on the special equipment and special reactor design used, for example, as shown in FIG. 1 (one or more packed bed towers) being representative for performing reactions in a counter-current reactor system, in particular in a loop reactor system, or in a counter-current (loop) system (“inverse gas scrubber system”), and, for example, as shown in FIG. 2 or 3 being representative for performing reactions in a tube reactor system, a continuous flow reactor system, in a coil reactor system, or in a microreactor system.

In particular, the fluorine (F₂) concentration of the F₂-fluorination gas used in the fluorination process step can be different for a reactor design for performing reactions in a counter-current reactor system, for example, as shown in FIG. 1 (one or more packed bed towers) on the one hand, and for a reactor design for performing reactions in a microreactor system, for example, as shown in FIG. 2 or 3.

Direct Fluorinationin a Column Reactor, e.g., in a Counter-Current Reactor System:

Preferably, in the F₂-fluorination gas, used in the direct fluorination reaction, the following (F₂) concentration is adjusted when performing reactions in a counter-current reactor system, in particular in a loop reactor system, or in a counter-current (loop) system (“inverse gas scrubber system”).

Regarding the F₂ concentration in the F₂-fluorination gas composition it is noted that in case of a counter-current reactor system, in particular in a loop reactor system, or in a counter-current (loop) system (“inverse gas scrubber system”), for example, as shown in the FIG. 1, the direct fluorination process can be worked equally with inert gas diluted F₂ and concentrated F₂, respectively, since inert gas can escape overhead via the pressure control valve, without any problems, for example, without any hotspots etc. in the reactor, which hotspots would reducing selectivity and yield.

Thus, when performing direct fluorination reactions in a counter-current reactor system, in particular in a loop reactor system, or in a counter-current (loop) system (“inverse gas scrubber system”), the direct fluorination reactions can be performed over the whole wide range of fluorine (F₂) concentration in the F₂-fluorination gas, as given here before, that is a fluorine (F₂) concentration in the F₂-fluorination of from about 1% by volume of elemental fluorine (F₂) up to about almost 100% by volume of elemental fluorine (F₂), based on the total F₂-fluorination gas composition as 100% by volume. Accordingly, in this case the direct fluorination reactions can be performed, for example, (i) in the typical ranges of lower fluorine (F₂) concentration in the F₂-fluorination gas as given above, (ii) in the typical ranges of higher fluorine (F₂) concentration in the F₂-fluorination gas as given above, but as well (iii) in the ranges of middle fluorine (F₂) concentration in the F₂-fluorination gas such as, for example, of from about >30% by volume of elemental fluorine (F₂) up to about <85% by volume of elemental fluorine (F₂).

It goes without saying that a skilled person will understand that within any of the above given ranges any intermediate values and intermediate ranges can be selected, too.

Direct Fluorinationin a Continuous Flow Reactor System, e.g., Microreactor System:

Preferably, in the F₂-fluorination gas, used in the direct fluorination reaction, the following fluorine (F₂) concentration is adjusted when performing reactions in a tube reactor system, in a continuous flow reactor system, in a coil reactor system, or in a microreactor system, preferably in a microreactor system.

Regarding the F₂ concentration in the F₂-fluorination gas composition it is noted that in case of a tube reactor system, a continuous flow reactor system, a coil reactor system, or a microreactor system, preferably in a microreactor system, the direct fluorination reaction preferably is performed, within the above mentioned typical ranges of higher fluorine (F₂) concentration in the F₂-fluorination gas. Thus, the higher fluorine (F₂) concentration in the F₂-fluorination gas preferably applied in a tube reactor system, in a continuous flow reactor system, in a coil reactor system, or in a microreactor system, for example, are of from about 85% by volume of elemental fluorine (F₂) up to about almost 100% by volume of elemental fluorine (F₂), preferably of from about 90% by volume of elemental fluorine (F₂) up to about almost 100% by volume of elemental fluorine (F₂), based on the total F₂-fluorination gas composition as 100% by volume.

Furthermore, regarding the F₂-concentration in the F₂-fluorination gas composition it is noted that, whereas in case of a counter-current reactor system, in particular in a loop reactor system, or in a counter-current (loop) system (“inverse gas scrubber system”), the direct fluorination process can be worked equally with inert gas diluted F₂ and concentrated F₂, respectively, as already explained above, in contrast, when performing reactions in a tube reactor system, a continuous flow reactor system, in a coil reactor system, or in a microreactor system, it is highly recommendable and preferred to have as little or even (almost) no inert gas in the F₂-fluorination gas composition, as during performing a reaction in said tube reactor system, continuous flow reactor system, coil reactor system, or microreactor system, no gas can escape, i.e., the inert gases are disadvantageous, because they create bubbles in the channels of a microreactor system and thereby hinder the exchange of heat and causing occurrence of hot spots, which then also would reduce selectivity and yield.

Therefore, if before start of reaction in a microreactor system, the system is continuously floated with an inert gas purge, for example, nitrogen (N₂) inert gas purge, the before start of the direct fluorination reaction in a microreactor system the concentration of inert gas preferably is rapidly reduced once the feeding of raw materials has started, to adjusted the F₂-concentration in the F₂-fluorination gas to the above said ranges of higher fluorine (F₂) concentration in the F₂-fluorination gas. A fast reduction of inert gas feed is essential as inert gas reduces sharply the heat exchange efficiency in the microchannel reactors.

It goes without saying that a skilled person will understand that within any of the above given ranges any intermediate values and intermediate ranges can be selected, too.

### BRIEF DESCRIPTION OF THE DRAWINGS

FIG. 1 shows the manufacture of PFP (perfluoropentane), both reaction steps (one step with F2 gas as fluorination agent, e.g., as the first step.

FIG. 2 shows the manufacture of PFP (perfluoropentane) in a microreactor system (two microreactors) in continuous manner.

FIG. 3 shows the manufacture of PFP (perfluoropentane) in a microreactor system, wherein the starting material compound is HFAA (hexafluoro acetyleacetone, with two microreactors in continuous manner.

### DETAILED DESCRIPTION OF THE INVENTION

As briefly described in the Summary of the Invention, and defined in the claims and further detailed by the following description and examples herein, the invention relates to a new industrial process for manufacturing of perfluoropentane (PFP), and/or of the compound perfluorinated 4-methylbutyrolactone, which is a suitable intermediate in the manufacture of perfluoropentane (PFP), involving reactions in liquid phase and performing reactions in a counter-current reactor system, in particular in a loop reactor system, or in a counter-current (loop) system (“inverse gas scrubber system”), as well as in a tube reactor system, a continuous flow reactor system, in a coil reactor system or in a microreactor system, preferably performing reactions in a counter-current reactor system or in a microreactor, respectively, as each described here under and in the claims.

In a first aspect, having exemplified the invention here before, the process of the present invention, more generally, is directed to a process for the manufacture of the compound PFP (perfluoropentane) having the formula (I),

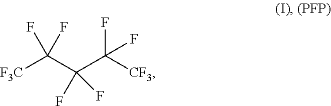 [SMILES: FC(F)(F)C(F)(F)C(F)(F)C(F)(F)C(F)(F)F]

wherein the compound PFP is manufactured starting from a starting material compound selected from the group consisting of (a) 4-methylbutyrolactone of formula (II),

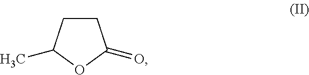

and of (b) an acetylacetone compound of formula (III),

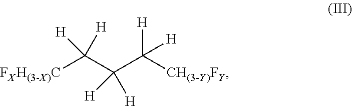

wherein in formula (III), independently of each other, X is an integer of 0 to 3, and Y is an integer of 0 to 3;
and
wherein the process is performed in a reactor or reactor system, resistant to elemental fluorine (F₂) and to hydrogen fluoride (1F), and the process comprises the steps of:
(i) a direct fluorination reaction with elemental fluorine (F2) as gaseous fluorination agent, and
(ii) a fluorination reaction with a fluorination agent selected from the group consisting of SF4 (sulfur tetrafluoride), commercial fluorinating agents like DAST, Deoxo-Fluor, Xtal-Fluor (M/E), Fluorlead and PhenoFluor, preferably SF4 as the fluorination agent;
and wherein
(A) either if the starting material is (a) 4-methylbutyrolactone of formula (II), the first process step is the direct fluorination reaction (i) with elemental fluorine (F2) as gaseous fluorination agent to yield a fluorinated intermediate compound (A); and the second process step is the fluorination reaction (ii) with a fluorination agent selected from the group consisting of SF4, commercial fluorinating agents like DAST, Deoxo-Fluor, Xtal-Fluor (M/E), Fluorlead and PhenoFluor, preferably SF4 as the fluorination agent, wherein the fluorinated intermediate compound (A) is further fluorinated to yield the compound PFP (perfluoropentane) having the formula (I);
(B) or if the starting material is (b) the acetylacetone compound of formula (III), the first process step is the fluorination reaction (ii) with a fluorination agent selected from the group consisting of SF4, commercial fluorinating agents like DAST, Deoxo-Fluor, Xtal-Fluor (M/E), Fluorlead and PhenoFluor, preferably SF4 as the fluorination agent to yield a fluorinated intermediate compound (B); and the second process step is the direct fluorination reaction (i) with elemental fluorine (F2) as gaseous fluorination agent, wherein the fluorinated intermediate compound (B) is further fluorinated to yield the compound PFP (perfluoropentane) having the formula (I).

Secondly, having exemplified the invention here before, the invention also pertains to a process for the manufacture of the compound PFP (perfluoropentane) having the formula (I), wherein the compound PFP is manufactured starting from the starting material compound (a) 4-methylbutyrolactone of formula (II).

Thirdly, having exemplified the invention here before, the invention furthermore pertains to a process for the manufacture of the compound PFP (perfluoropentane) having the formula (I), wherein the compound PFP is manufactured starting from the starting material compound which is (b) an acetylacetone compound of formula (III).

Fourthly, having exemplified the invention here before, the invention furthermore pertains to a process for the manufacture of the compound PFP (perfluoropentane) having the formula (I), wherein the starting material compound of (b) an acetylacetone is selected from the group consisting of acetylacetone, 1,1,1-trifluoro-2,4-pentanedione (trifluoro acetylacetone; TFAA) and hexafluoro acetylacetone (HFAA).

Fifthly, having exemplified the invention here before, the invention furthermore pertains to a process for the manufacture of the compound PFP (perfluoropentane) having the formula (I), wherein the process step of the fluorination reaction (ii) is performed with SF₄ as the fluorination agent.

Sixthly, having exemplified the invention here before, the invention furthermore pertains to a process for the manufacture of the compound PFP (perfluoropentane) having the formula (I), according to claim 1, wherein the compound PFP is manufactured starting from the starting material compound (a) 4-methylbutyrolactone of formula (II); and wherein the process step of the fluorination reaction (ii) is performed with SF₄ as the fluorination agent.

In a second aspect, having exemplified the invention here before, the process of the present invention, generally is also directed to a process for the manufacture of the perfluorinated lactone compound having the formula (IV), which is perfluorinated 4-methylbutyrolactone,

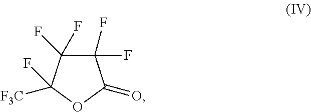 [SMILES: O=C1OC(F)(C(F)(F)F)C(F)(F)C1(F)F]

wherein the perfluorinated 4-methylbutyrolactone compound is manufactured starting from a starting material compound of formula (II), which is 4-methylbutyrolactone,

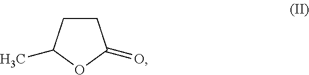

and
wherein the process is performed in a reactor or reactor system, resistant to elemental fluorine (F₂) and to hydrogen fluoride (1F), and the process comprises a direct fluorination reaction (i) with elemental fluorine (F₂) as gaseous fluorination agent, to yield the perfluorinated 4-methylbutyrolactone compound of formula (IV), which is perfluorinated 4-methylbutyrolactone.

In relation to said second aspect, the present invention also is directed to the perfluorinated lactone compound having the formula (IV), which is perfluorinated 4-methylbutyrolactone,

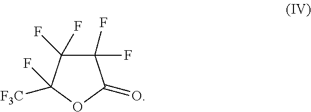 [SMILES: O=C1OC(F)(C(F)(F)F)C(F)(F)C1(F)F]

Furthermore, in relation to said second aspect, the present invention also is directed to the use of a perfluorinated lactone compound having the formula (IV), which is perfluorinated 4-methylbutyrolactone,

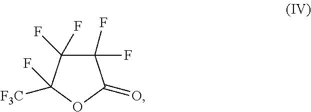 [SMILES: O=C1OC(F)(C(F)(F)F)C(F)(F)C1(F)F]

in the manufacture the of the compound PFP (perfluoropentane) having the formula

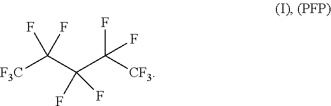 [SMILES: FC(F)(F)C(F)(F)C(F)(F)C(F)(F)C(F)(F)F]

In a third aspect, having exemplified the invention here before, the process of the present invention, more generally, is directed to a process for the manufacture of the compound PFP (perfluoropentane) having the formula (I),

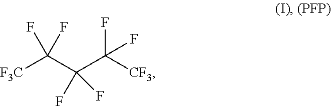 [SMILES: FC(F)(F)C(F)(F)C(F)(F)C(F)(F)C(F)(F)F]

wherein the compound PFP is manufactured starting from a perfluorinated lactone compound having the formula (IV), which is perfluorinated 4-methylbutyrolactone,

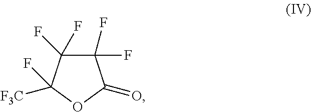 [SMILES: O=C1OC(F)(C(F)(F)F)C(F)(F)C1(F)F]

wherein the process is performed in a reactor or reactor system, resistant to elemental fluorine (F₂) and to hydrogen fluoride (HF), and the process comprises a fluorination reaction (ii) with a fluorination agent selected from the group consisting of SF₄, commercial fluorinating agents like DAST, Deoxo-Fluor, Xtal-Fluor (M/E), Fluorlead and PhenoFluor, preferably SF₄ as the fluorination agent, to yield the compound PFP (perfluoropentane) having the formula (I).

In another aspect the present invention also is directed to a process for the manufacture of the compound PFP (perfluoropentane) having the formula (I), wherein the fluorination reaction (ii) is performed with SF₄ as the fluorination agent.

In still another aspect the present invention also is directed to a process for the manufacture of the compound PFP (perfluoropentane) having the formula (I), as described above, wherein the fluorination reaction (ii) is performed with SF₄ as the fluorination agent, and in HF (hydrogen fluoride) as solvent.

In further aspect the present invention also is directed to a process for the manufacture of the compound PFP (perfluoropentane) having the formula (I), wherein the fluorination reaction (ii) is performed with SF₄ as the fluorination agent, and in HF (hydrogen fluoride) as solvent, and in the presence of a Lewis acid.

In still further aspect the present invention also is directed to a process for the manufacture of the compound PFP (perfluoropentane) having the formula (I), according to claim 13, wherein the Lewis acid is selected from the group consisting of Lewis acids like TiClₙF₄₋ₙ, SnClnF₄₋ₙ, SbClₘF₅₋ₘ, wherein n denotes an integer of 0 to 4, and m denotes an integer of 0 to 5.

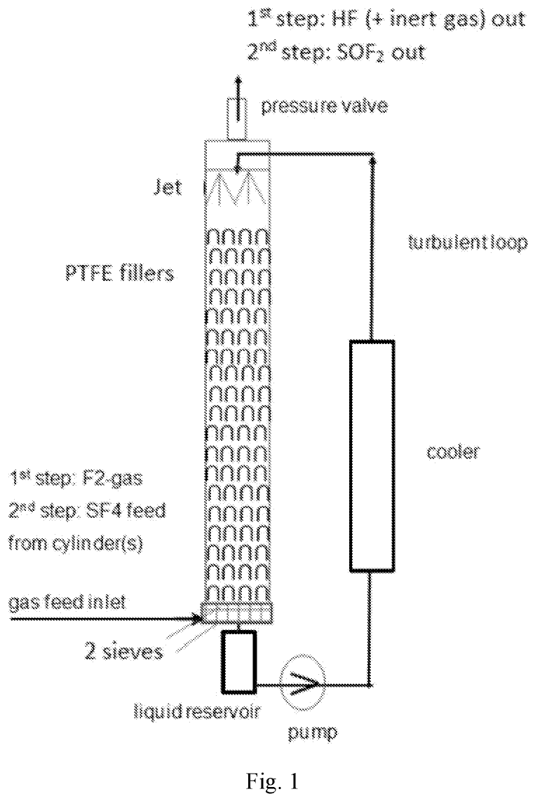

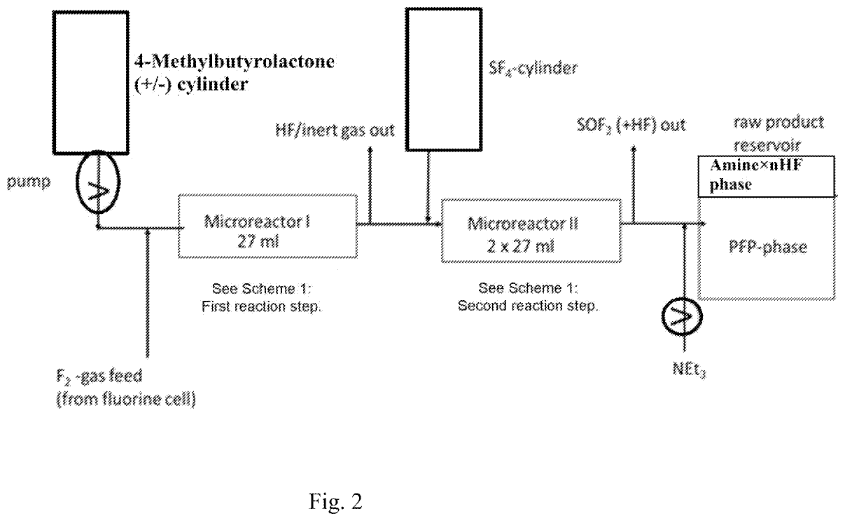

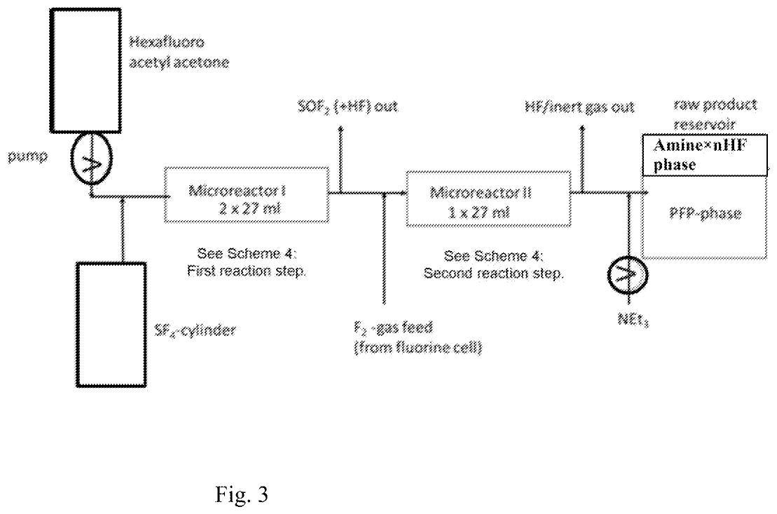

The reaction steps, direct fluorination reaction with F₂-gas the fluorination agent and fluorination reaction with SF₄ as the fluorination agent (and, if applicable, involving the lactone ring opening), in the processes according to the present invention, described herein and in the claims, may be performed in various reactor designs. Example reactor designs include, a loop reactor system, a counter-current (loop) system (“inverse gas scrubber system”), a microreactor system (may include one or more), and coil reactor design. Particular reactor designs are shown in the FIG. 1 (gas scrubber system, counter-current [loop] system), FIGS. 2 and 3 (microreactor systems). Further, the direct fluorination step with F₂-gas the fluorination agent in the process of the invention may be performed in a batch or in a continuous manner, respectively. Further, any of the direct fluorination step with F₂-gas the fluorination agent, and the fluorination reaction with SF₄ as the fluorination agent (and, if applicable, involving the lactone ring opening) in the process of the invention may be performed in a batch or in a continuous manner, respectively.

A preferred reactor used in any one of the steps, e.g., in one or more or in all steps of direct fluorination step with F₂-gas the fluorination agent and fluorination reaction with SF₄ as the fluorination agent (and, if applicable, involving the lactone ring opening), of the present invention independently is a microreactor system.

In the present invention, the reactor may be also a loop reactor system, a counter-current (loop) system (“inverse gas scrubber system”), but preferably the reactor is microreactor system. See FIG. 1 (gas scrubber system, counter-current [loop] system), or see FIGS. 2 and 3 (microreactor system), respectively.

In case of a continuous manner process, i.e., when the continuous process according to the invention is performed in any one of the steps of direct fluorination step with F₂-gas the fluorination agent and fluorination reaction with SF₄ as the fluorination agent (and, if applicable, involving the lactone ring opening), independently, in the present invention the reactor system is a microreactor system as described herein and in the claims, and used in continuous operating manner.

In case of a batch manner process, i.e., when the batch process according to the invention is performed in any one of the steps of direct fluorination step with F₂-gas the fluorination agent and fluorination reaction with SF₄ as the fluorination agent (and, if applicable, involving the lactone ring opening), independently, the batch process according to the invention can also be performed in a counter-current system, preferably as described herein and in the claims, in batch operating manner.

The invention also relates to a direct fluorination step with F₂-gas the fluorination agent and fluorination reaction with SF₄ as the fluorination agent (and, if applicable, involving the lactone ring opening), as each described herein and in the claims, optionally either operated in a batch manner or operated in a continuous manner, for the manufacture of the compound PFP (perfluoropentane), and/or of the compound perfluorinated 4-methylbutyrolactone, i.e., the precursor or intermediate compound of PFP (perfluoropentane), respectively, as each defined herein and in the claims, wherein the reaction is carried out in at least one of the steps of direct fluorination step with F₂-gas the fluorination agent and fluorination reaction with SF₄ as the fluorination agent (and, if applicable, involving the lactone ring opening) as a continuous processes, wherein the continuous process is performed in at least one continuous flow reactor with upper lateral dimensions of about ≤5 mm, or of about ≤4 mm, preferably in at least one microreactor.

The invention also relates to a process, as described herein, optionally either operated in a batch manner or operated in a continuous manner, for the manufacture of PFP (perfluoropentane), and/or of the compound perfluorinated 4-methylbutyrolactone, i.e., the precursor or intermediate compound of PFP (perfluoropentane), characterized in that said reaction step is performed in a SiC-reactor.

The invention also relates to a process, as described herein, optionally either operated in a batch manner or operated in a continuous manner, for the manufacture of PFP (perfluoropentane), and/or of the compound perfluorinated 4-methylbutyrolactone, i.e., the precursor or intermediate compound of PFP (perfluoropentane), characterized in that said step is performed in a nickel-reactor (Ni-reactor) or in a reactor with an inner surface with high nickel-content (Ni-content).

As already described above, when performing direct fluorination reactions in a counter-current reactor system, in particular in a loop reactor system, or in a counter-current (loop) system (“inverse gas scrubber system”), the direct fluorination reactions can be performed over the whole wide range of fluorine (F₂) concentration in the F₂-fluorination gas of from about 1% by volume of elemental fluorine (F₂) up to about almost 100% by volume of elemental fluorine (F₂), based on the total F₂-fluorination gas composition as 100% by volume.

In this aspect, for example, the invention pertains to a process for the manufacture of the compound PFP (perfluoropentane), and/or of perfluorinated 4-methylbutyrolactone compound, i.e., the precursor or intermediate compound of PFP (perfluoropentane), wherein the direct fluorination reaction is performed in a counter-current reactor system, in particular in a loop reactor system, or in a counter-current (loop) system (“inverse gas scrubber system”), and
wherein the fluorine (F₂) concentration in the F₂-fluorination gas is in a range of from about 1% by volume of elemental fluorine (F₂) up to about almost 100% by volume of elemental fluorine (F₂), based on the total F₂-fluorination gas composition as 100% by volume;
preferably wherein
(i) the fluorine (F2) concentration in the F2-fluorination gas is in a range of from about 1% by volume of elemental fluorine (F2) up to about 30% by volume of elemental fluorine (F2), more preferably of from about 5% by volume of elemental fluorine (F2) up to about 25% by volume of elemental fluorine (F2), even more preferably of from about 5% by volume of elemental fluorine (F2) up to about 20% by volume of elemental fluorine (F2), each range based on the total F2-fluorination gas composition as 100% by volume; or
(ii) the fluorine (F2) concentration in the F2-fluorination gas is in a range of from about 85% by volume of elemental fluorine (F2) up to about almost 100% by volume of elemental fluorine (F2), more preferably of from about 90% by volume of elemental fluorine (F2) up to about almost 100% by volume of elemental fluorine (F2), based on the total F2-fluorination gas composition as 100% by volume.

Accordingly, when performing direct fluorination reactions in a counter-current reactor system, in particular in a loop reactor system, or in a counter-current (loop) system (“inverse gas scrubber system”), in one aspect the invention also pertains to a process as defined above, for the manufacture of the compound PFP (perfluoropentane), and/or of perfluorinated 4-methylbutyrolactone compound, i.e., the precursor or intermediate compound of PFP (perfluoropentane), wherein the lower fluorine (F₂) concentration in the F₂-fluorination gas is applied, and wherein the fluorination gas in the direct fluorination reaction step is elemental fluorine (F₂) diluted in one or more inert gases, and wherein the elemental fluorine (F₂) is present in the fluorination gas in a concentration in a range of from about 1% up to about 30% by volume of elemental fluorine (F₂), preferably of from about 5% up to about 25% by volume of elemental fluorine (F₂), more preferably of from about 5% up to about 20% by volume of elemental fluorine (F₂), each range based on the total F₂-fluorination gas composition as 100% by volume. Even more preferably, when performing reactions in said counter-current reactor system, in particular loop reactor system, or counter-current (loop) system (“inverse gas scrubber system”), the fluorination gas in the direct fluorination reaction step is elemental fluorine (F₂) diluted in one or more inert gases, and the elemental fluorine (F₂) is present in the fluorination gas in a concentration in a range of from about 5% up to about 15% by volume of elemental fluorine (F₂), still more preferably in a range of about 8% up to about 15% by volume of elemental fluorine (F₂), and most preferably in a range of about 8% up to about 12% by volume of elemental fluorine (F₂), e.g., the elemental fluorine (F₂) is present in the fluorination gas in a concentration of about 10% by volume (e.g., 10±2% by volume, or 10±1% by volume, respectively). It goes without saying that a skilled person will understand that within any of the above given ranges any intermediate values and intermediate ranges can be selected, too.

Accordingly, when performing direct fluorination reactions in a counter-current reactor system, in particular in a loop reactor system, or in a counter-current (loop) system (“inverse gas scrubber system”), in another aspect, the invention also pertains to a process as defined above, for the manufacture of the compound PFP (perfluoropentane), and/or of perfluorinated 4-methylbutyrolactone compound, i.e., the precursor or intermediate compound of PFP (perfluoropentane), wherein the higher fluorine (F₂) concentration in the F₂-fluorination gas is applied, and wherein the elemental fluorine (F₂) is present in the fluorination gas in a concentration in a range of from about 85% up to about almost 100% (as defined herein above) by volume of elemental fluorine (F₂), most preferably of from about 90% by volume of elemental fluorine (F₂) up to about almost 100% (as defined herein above) by volume of elemental fluorine (F₂), based on the total F₂-fluorination gas composition as 100% by volume. Even more preferably, when performing reactions in said counter-current reactor system, in particular loop reactor system, or counter-current (loop) system (“inverse gas scrubber system”), the F₂-fluorination gas used in the direct fluorination process step of the invention, for example, is a fluorine (F₂) gas only to some extent diluted in an inert gas (together then they constitute the F₂-fluorination gas), with fluorine (F₂) concentrations in ranges, for example, with a maximum concentration of up to about almost 100% by volume of elemental fluorine (F₂), in the range of from about 85% by volume, in particular in the range of from about 90% by volume or in particular in the range of from about 92% by volume of elemental fluorine (F₂), especially in the range of from about 94% by volume; each given range based on the fluorine (F₂) gas and the inert gas as 100% by volume, i.e., based on the total F₂-fluorination gas composition as 100% by volume.

In another aspect, when performing direct fluorination reactions in a counter-current reactor system, in particular in a loop reactor system, or in a counter-current (loop) system (“inverse gas scrubber system”), the invention also pertains to a process as defined above, for the manufacture of the compound PFP (perfluoropentane), and/or of perfluorinated 4-methylbutyrolactone compound, i.e., the precursor or intermediate compound of PFP (perfluoropentane), wherein the higher fluorine (F₂) concentration in the F₂-fluorination gas is applied, and wherein, in a very practical range, for example, in particular if the F₂-fluorination gas is derived from an F₂-electrolysis reactor (fluorine cell), purified or unpurified, and wherein the fluorine (F₂) gas from the F₂-electrolysis reactor (fluorine cell) is only to some extent diluted in an inert gas (together then they constitute the F₂-fluorination gas), with fluorine (F₂) in a concentration within a range of from about 92% by volume of elemental fluorine (F₂) up to about 99% by volume of elemental fluorine (F₂), and most preferably in a very practical range of from about 94% by volume to about 99% by volume; each given range based on the fluorine (F₂) gas and the inert gas as 100% by volume, i.e., based on the total F₂-fluorination gas composition as 100% by volume.

It goes without saying that a skilled person will understand that within any of the above given ranges any intermediate values and intermediate ranges can be selected, too.

In still another aspect, the invention pertains to a process as defined here before, for the manufacture of the compound PFP (perfluoropentane), and/or of perfluorinated 4-methylbutyrolactone compound, i.e., the precursor or intermediate compound of PFP (perfluoropentane), wherein the liquid reaction medium is circulated in a loop in a (closed) column reactor, and wherein the loop is operated with a circulation velocity in the range of from about 1,000 l/h to about 2,000 l/h, preferably in the range of from about 1,250 l/h to about 1,750 l/h; more preferably wherein the loop is operated with a circulation velocity in the range of from about 1,500 l/h±200 l/h; even more preferably wherein the loop is operated with a circulation velocity in the range of from about 1,500 l/h±100 l/h; and most preferably wherein the loop is operated with a circulation velocity in the range of from about 1,500 l/h±50 l/h.

For example, in said still another aspect of the invention as defined here before, pertains to a process as defined here before, for the manufacture of the compound PFP (perfluoropentane), and/or of perfluorinated 4-methylbutyrolactone compound, i.e., the precursor or intermediate compound of PFP (perfluoropentane), wherein for the reaction the (closed) column reactor is equipped with at least one of the following:
(i) at least one heat exchanger (system), at least one liquid reservoir, with inlet and outlet for, and containing the liquid reaction medium,
e.g., initially comprising or consisting of the starting material compound, or as the reaction proceeds increasingly comprising or consisting of the product compound PFP (perfluoropentane), and/or of perfluorinated 4-methylbutyrolactone compound, i.e., the precursor or intermediate compound of PFP (perfluoropentane), or any of the other above said 2,4-diketone starting materials or intermediate compounds derived therefrom;
(ii) a pump for pumping and circulating the liquid reaction medium;
(iii) one or more (nozzle) jets, preferably wherein the one or more (nozzle) jets are placed at the top of the column reactor, for spraying the circulating reaction medium into the (closed) column reactor;
(iv) optionally one or more sieves, preferably two sieves, preferably the one or more sieves placed at the bottom of the (closed) column reactor;
(v) and at least one gas outlet equipped with a pressure valve, and at least one outlet for withdrawing the product compound PFP (perfluoropentane), and/or of perfluorinated 4-methylbutyrolactone compound, i.e., the precursor or intermediate compound of PFP (perfluoropentane), or any of the other above said 2,4-diketone starting materials or intermediate compounds derived therefrom, from the (closed) column reactor.

In an aspect of the invention, wherein the direct fluorination reaction with F₂-gas as the fluorination agent, and/or the fluorination reaction with SF₄ as the fluorination agent (and, if applicable, involving the lactone ring opening) is carried out in a (closed) column reactor, the invention pertains to a process as defined here before, for the manufacture of the compound PFP (perfluoropentane), and/or of perfluorinated 4-methylbutyrolactone compound, i.e., the precursor or intermediate compound of PFP (perfluoropentane), wherein the column reactor is a packed bed tower reactor, preferably a packed bed tower reactor is packed with fillers resistant to the reactants and especially resistant to elemental fluorine (F₂) and to hydrogen fluoride (HF) such as, e.g., with Raschig fillers, E-TFE fillers, and/or HF-resistant metal fillers, e.g., Hastelloy metal fillers, and/or (preferably) HDPTFE-fillers, more preferably wherein the packed bed tower reactor is a gas scrubber system (tower) which is packed with any of the before mentioned HF-resistant Hastelloy metal fillers and/or HDPTFE-fillers, and preferably with HDPTFE-fillers.

In yet a further aspect the invention pertains to a process as defined here before, for the manufacture of the compound PFP (perfluoropentane), and/or of perfluorinated 4-methylbutyrolactone compound, i.e., the precursor or intermediate compound of PFP (perfluoropentane), wherein the direct fluorination reaction with F₂-gas as the fluorination agent, and/or the fluorination reaction with SF₄ as the fluorination agent (and, if applicable, involving the lactone ring opening) is carried out in at least one step in a continuous flow reactor with upper lateral dimensions of about ≤5 mm, or of about ≤4 mm, more preferably in at least one step in a microreactor;
still more preferably wherein the direct fluorination reaction with F₂-gas as the fluorination agent, and/or the fluorination reaction with SF₄ as the fluorination agent (and, if applicable, involving the lactone ring opening) is carried out in at least in one step as a continuous processes, wherein the continuous process is performed in at least one continuous flow reactor with upper lateral dimensions of about ≤5 mm, or of about ≤4 mm;
even more preferably wherein the direct fluorination reaction with F₂-gas as the fluorination agent, and/or the fluorination reaction with SF₄ as the fluorination agent (and, if applicable, involving the lactone ring opening) is carried out in at least in one step as a continuous processes, wherein the continuous process is performed in at least one microreactor.

In still a further aspect the invention pertains to a process as defined here before, for the manufacture of the compound PFP (perfluoropentane), and/or of perfluorinated 4-methylbutyrolactone compound, i.e., the precursor or intermediate compound of PFP (perfluoropentane), characterized in that prior to starting any of the process steps of direct fluorination reaction with F₂-gas as the fluorination agent, and/or the fluorination reaction with SF₄ as the fluorination agent (and, if applicable, involving the lactone ring opening) one or more of the reactors used, preferably each and any of the reactors used, are purged with an inert gas or a mixture of inert gases, preferably with He (helium) and/or N₂ (nitrogen) as the inert gas, more preferably with N₂ (nitrogen) as the inert gas.

In a particular aspect the invention pertains to a process as defined here before, for the manufacture of the compound PFP (perfluoropentane), and/or of perfluorinated 4-methylbutyrolactone compound, i.e., the precursor or intermediate compound of PFP (perfluoropentane), characterized in that in the fluorination reaction step (A) the reaction is performed in a SiC-reactor; preferably in that in the fluorination reaction step (A) the reaction is performed in a SiC-microreactor.

In another particular aspect the invention pertains to a process as defined here before, for the manufacture of the compound PFP (perfluoropentane), and/or of perfluorinated 4-methylbutyrolactone compound, i.e., the precursor or intermediate compound of PFP (perfluoropentane), the reaction is performed in a nickel-reactor (Ni-reactor) or in a reactor with an inner surface with high nickel-content (Ni-content).

In a further particular aspect the invention pertains to a process as defined here before, for the manufacture of the compound PFP (perfluoropentane), and/or of perfluorinated 4-methylbutyrolactone compound, i.e., the precursor or intermediate compound of PFP (perfluoropentane), characterized in that, independently, the product yielding from direct fluorination reaction with F₂-gas as the fluorination agent, and/or the fluorination reaction with SF₄ as the fluorination agent (and, if applicable, involving the lactone ring opening) are subjected to distillation.

In still another aspect, the invention pertains also to any one of the above defined processes for the manufacture of the compound PFP (perfluoropentane), and/or of perfluorinated 4-methylbutyrolactone compound, i.e., the precursor or intermediate compound of PFP (perfluoropentane), characterized in that the reaction is performed in a nickel-reactor (Ni-reactor) or in a reactor with an inner surface with high nickel-content (Ni-content). Preferably, in the context of the present invention the term “high nickel-content” means a nickel (Ni) content of at least 50% in the metal alloy the nickel-reactor is made of. Particularly preferred is a nickel-reactor made out of Hastelloy C4 nickel alloy. The Hastelloy C4 nickel alloy is known in the state of the art to be a nickel alloy comprising a combination of chromium with high molybdenum content. Such Hastelloy C4 nickel alloy shows exceptional resistance to a large number of chemical media such as contaminated, reducing mineral acids, chlorides and organic and inorganic media contaminated with chloride.

Hastelloy C4 nickel alloy is commercially available, for example, under the tradenames Nicrofer® 6616 hMo or Hastelloy C-4®, respectively. The density of Hastelloy C4 nickel alloy is 8.6 g/cm³, and the melting temperature range is 1335 to 1380° C.

Due to its special chemical composition of C4, the Hastelloy C4 nickel alloy has good structural stability and high resistance to sensitization.

The chemical composition of Hastelloy C4 (nickel alloy), for example, is in the following Table 1, wherein the nickel (Ni) content is at least 50% in the metal alloy, and the nickel (Ni) content is adding up the Hastelloy C4 nickel alloy compositions to a total of 100% metal alloy.

TABLE 1
Chemical composition of Hastelloy C4 (nickel alloy).

| C | Si | Mn | P | S | Cr | Mo | Co | Fe | Ti |
|---|---|---|---|---|---|---|---|---|---|
| % | ≤% | ≤% | ≤% | ≤% | % | % | ≤% | % | % |
| 0-0.009 | 0-0.05 | 0-1.0 | 0-0.02 | 0-0.01 | 14.5-17.5 | 14.0-17.0 | 0-2.0 | 0-3 | 0-0.7 |

and nickel (Ni) as the remainder for adding up to 100% metal alloy.

Batch Process:

The invention also may pertain to a process for the manufacture of the compound PFP (perfluoropentane), and/or of perfluorinated 4-methylbutyrolactone compound, i.e., the precursor or intermediate compound of PFP (perfluoropentane), comprising a particular process step which is performed batchwise, preferably wherein the batchwise process step is carried out in a column reactor. Although, in the following column reactor setting the process is described as a batch process, optionally the process can be performed in the said column reactor setting also as a continuous process. In case of a continuous process in the said column reactor setting, then, it goes without saying, the additional inlet(s) and outlet(s) are foreseen, for feeding the starting compound and withdrawing the product compound, respectively, and/or if desired any intermediate compound.

If the invention pertains to a batchwise process, preferably wherein the batchwise process is carried out in a column reactor, the process for manufacturing of the compound PFP (perfluoropentane), and/or of perfluorinated 4-methylbutyrolactone compound, i.e., the precursor or intermediate compound of PFP (perfluoropentane), most preferably the reaction is carried out in a (closed) column reactor (system), wherein the liquid medium comprising or consisting of a liquid starting compound, e.g., the 4-methylbutyrolactone compound or perfluorinated 4-methylbutyrolactone compound, respectively, as a liquid medium is circulated in a loop; preferably wherein the loop in the column reactor is operated with a circulation velocity of from 1,500 l/h to 5,000 l/h, more preferably of from 3,500 l/h to 4,500 l/h.

If the invention pertains to such a batchwise process, the process for manufacturing of the compound PFP (perfluoropentane), and/or of perfluorinated 4-methylbutyrolactone compound, i.e., the precursor or intermediate compound of PFP (perfluoropentane), according to the invention can be carried out such that the mentioned liquid medium is circulated in the column reactor in a turbulent stream or in laminar stream, preferably in a turbulent stream.

In general, a gaseous starting compound, e.g., the F₂-fluorination gas, respectively, is fed into the loop in accordance with the required stoichiometry for the targeted product compound and/or if desired any intermediate compound, and adapted to the reaction rate.

For example, the said process for the manufacture of the compound PFP (perfluoropentane), and/or of perfluorinated 4-methylbutyrolactone compound, i.e., the precursor or intermediate compound of PFP (perfluoropentane), according to the invention, may be performed, e.g., batchwise, wherein the column reactor is equipped with at least one of the following: at least one cooler (system), at least one liquid reservoir for the liquid medium comprising or consisting of a liquid starting compound, a pump (for pumping/circulating the liquid medium), one or more (nozzle) jets, preferably placed at the top of the column reactor, for spraying the circulating medium into the column reactor, one or more feeding inlets for introducing a gaseous starting compound, e.g., F₂-fluorination gas, optionally one or more sieves, preferably two sieves, preferably the one or more sieves placed at the bottom of the column reactor, and at least one gas outlet equipped with a pressure valve.

Accordingly, the process for manufacturing of the compound PFP (perfluoropentane), and/or of perfluorinated 4-methylbutyrolactone compound, i.e., the precursor or intermediate compound of PFP (perfluoropentane), according to the invention, can be performed in column reactor which is equipped with at least one of the following:
(i) at least one cooler (system), at least one liquid reservoir, with inlet and outlet for, and containing the liquid medium comprising or consisting of a starting compound; preferably the compound 4-methylbutyrolactone or perfluorinated 4-methylbutyrolactone compound, respectively;
(ii) a pump for pumping and circulating the liquid medium in the column reactor;
(iii) one or more (nozzle) jets, preferably wherein the one or more (nozzle) jets are placed at the top of the column reactor, for spraying the circulating liquid medium into the column reactor;
(iv) one or more feeding inlets for introducing a gaseous compound, e.g., inert gas or a F2-fluorination gas, respectively into the column reactor;
(v) optionally one or more sieves, preferably two sieves, preferably the one or more sieves placed at the bottom of the column reactor;
(vi) and at least one gas outlet equipped with a pressure valve, and at least one outlet for withdrawing the product compound, respectively, and/or if desired any intermediate compound.

In one embodiment, the process for manufacturing of the compound PFP (perfluoropentane), and/or of perfluorinated 4-methylbutyrolactone compound, i.e., the precursor or intermediate compound of PFP (perfluoropentane), according to the invention can be performed in a column reactor which is a packed bed tower reactor, preferably a packed bed tower reactor which is packed with fillers (the terms “filler” and “filling”, are meant synonymously in the context of the invention) resistant to the reactants and especially resistant to hydrogen fluoride (HF). Fillers resistant to the reactants and especially resistant to hydrogen fluoride (HF) suitable in the context of the present invention are in particular HF-resistant plastic fillers and/or 1F-resistant metal fillers. For example, under certain circumstances the packed bed tower reactor may be packed with stainless steel (1.4571) fillers, but stainless steel (1.4571) fillers are less suitable than other fillers mentioned herein after, because of possible risk of (minor) traces of humidity in the reactor system. Preferably, for example, in the invention the packed bed tower reactor is packed with fillers resistant to the reactants and especially resistant to hydrogen fluoride (HF) such as, e.g., with Raschig fillers, E-TFE fillers, and/or 1F-resistant metal fillers, e.g., Hastelloy metal fillers, and/or (preferably) HDPTFE-fillers, more preferably wherein the packed bed tower reactor is a gas scrubber system (tower) which is packed with any of the before mentioned 1F-resistant Hastelloy metal fillers and/or HDPTFE-fillers, and preferably with HDPTFE-fillers. The term “HDPTFE-filler” sometimes is shortened to the term “PTFE-filler”. Hence, the term “PTFE-filler” is synonymous to “HDPTFE-filler”.

In a further embodiment, the process for manufacturing of the compound PFP (perfluoropentane), and/or of perfluorinated 4-methylbutyrolactone compound, i.e., the precursor or intermediate compound of PFP (perfluoropentane), according to the invention, the reaction is carried out with a counter-current flow of the circulating liquid medium comprising or consisting of the liquid starting compound and of the F₂-fluorination gas, respectively, that are fed into the column reactor.

The pressure valve functions to keep the pressure, as required in the reaction, and to release any effluent gas, e.g. inert carrier gas contained in the fluorination gas, if applicable together with any hydrogen halogenide gas released from the reaction.

The said process for manufacturing of the compound PFP (perfluoropentane), and/or of perfluorinated 4-methylbutyrolactone compound, i.e., the precursor or intermediate compound of PFP (perfluoropentane), according to the invention, may be performed, e.g., batchwise, such that in the said process the column reactor is a packed bed tower reactor as mentioned before, preferably a packed bed tower reactor which is packed with HDPTFE-fillers or PTFE-fillers”, respectively.

The packed tower according to FIG. 1 can have a diameter of 100 or 200 mm (depending on the circulating flow rate and scale) made out of Hastelloy C4 (nickel alloy) (known to the person skilled in the art), and has a length of 3 meters for the 100 mm and a length of 6 meters for the 200 mm diameter tower (latter if higher capacities are needed). The tower made out of Hastelloy is filled either with any of the fillings as mentioned before, or with the preferred HDPTFE-fillers or PTFE-fillers, respectively, each of 10 mm diameter as commercially available. The size of fillings is quite flexible. The type of fillings is also quite flexible, within the boundaries of properties as stated herein above, i.e., the HDPTFE-fillers (or PTFE-fillings, respectively) were used in the trials disclosed hereunder in Example 2, and showed same performance, not causing much pressure reduction (pressure loss) while feeding any gaseous (starting) compound in counter-current manner.

Methods in a Continuous Flow Reactor System, e.g., Microreactor System:

The methods of the present invention as preferably described with microreactor are applicable to a continuous flow reactor system, as well as to a tube reactor system, and also applicable also to variants with coiled reactor system.

As already described above, when performing direct fluorination reactions in a tube reactor system, in a continuous flow reactor system, in a coil reactor system, or in a microreactor system, preferably in a microreactor system, preferably the higher fluorine (F₂) concentration in the F₂-fluorination gas (as defined above) is adjusted when performing the fluorination reactions.

In this aspect, for example, the invention pertains to a process for the manufacture of the compound PFP (perfluoropentane), and/or of perfluorinated 4-methylbutyrolactone compound, i.e., the precursor or intermediate compound of PFP (perfluoropentane), wherein the direct fluorination reaction is performed in a tube reactor system, in a continuous flow reactor system, in a coil reactor system, or in a microreactor system, preferably in a microreactor system, and wherein the fluorine (F₂) concentration in the F₂-fluorination gas is in a range of from about 85% by volume of elemental fluorine (F₂) up to about almost 100% by volume of elemental fluorine (F₂), more preferably of from about 90% by volume of elemental fluorine (F₂) up to about almost 100% by volume of elemental fluorine (F₂), based on the total F₂-fluorination gas composition as 100% by volume.

Accordingly, when performing direct fluorination reactions in a tube reactor system, in a continuous flow reactor system, in a coil reactor system, or in a microreactor system, preferably in a microreactor system, the F₂-fluorination gas used in the fluorination process step (A) of the invention, for example, is a fluorine (F₂) gas only to some extent diluted in an inert gas (together then they constitute the F₂-fluorination gas), with fluorine (F₂) concentrations in ranges, for example, with a maximum concentration of up to about almost 100% by volume of elemental fluorine (F₂), in the range of from about 85% by volume, in particular in the range of from about 90% by volume or in particular in the range of from about 92% by volume of elemental fluorine (F₂), especially in the range of from about 94% by volume; each given range based on the fluorine (F₂) gas and the inert gas as 100% by volume, i.e., based on the total F₂-fluorination gas composition as 100% by volume.

Accordingly, when performing direct fluorination reactions in a tube reactor system, in a continuous flow reactor system, in a coil reactor system, or in a microreactor system, preferably in a microreactor system, the said direct fluorination process step of the invention, for example, a fluorine (F₂) gas is only to some extent diluted in an inert gas (together then they constitute the F₂-fluorination gas), with fluorine (F₂) in a concentration more preferably within a range of from about 92% by volume to about almost 100% by volume, even more preferably within a range of from about 94% by volume to about almost 100% by volume, still more preferably in a very practical range, for example, in particular if the F₂-fluorination gas is derived from an F₂-electrolysis reactor (fluorine cell), purified or unpurified, of from about 92% by volume of elemental fluorine (F₂) up to about 99% by volume of elemental fluorine (F₂), and most preferably in a very practical range of from about 94% by volume to about 99% by volume; each given range based on the fluorine (F₂) gas and the inert gas as 100% by volume, i.e., based on the total F₂-fluorination gas composition as 100% by volume.

According to a preferred embodiment of the present invention, the compound PFP (perfluoropentane), and/or of perfluorinated 4-methylbutyrolactone compound, i.e., the precursor or intermediate compound of PFP (perfluoropentane) can also be prepared in a continuous manner. More preferably, the compound PFP (perfluoropentane), and/or the perfluorinated 4-methylbutyrolactone compound, i.e., the precursor or intermediate compound of PFP (perfluoropentane), are manufactured in microreactor reactor system.

Optionally, any intermediate in the process for manufacturing of the compound PFP (perfluoropentane), and/or of perfluorinated 4-methylbutyrolactone compound, i.e., the precursor or intermediate compound of PFP (perfluoropentane), according to the invention may be isolated and/or purified, and then such isolated and/or purified may be further processed, as desired. For example, the compound perfluorinated 4-methylbutyrolactone, which is a suitable intermediate in the manufacture of the compound PFP (perfluoropentane), may be isolated and/or purified. For example, the compound perfluorinated 4-methylbutyrolactone is prepared in a first microreactor by direct fluorination is optionally isolated and/or purified, and then the compound perfluorinated 4-methylbutyrolactone is transferred into another (second) microreactor to be further reacted in a second reaction step by fluorination reaction with SF₄ as the fluorination agent (and, if applicable, involving the lactone ring opening) to yield the compound PFP (perfluoropentane).

The intermediate compound perfluorinated 4-methylbutyrolactone produced in the mentioned first microreactor by direct fluorination, optionally may be isolated and/or purified, and then can also constitute the final product in isolated and/or purified form.

Alternatively, (intermediate) compound 1 perfluorinated 4-methylbutyrolactone produced in a first microreactor by perfluorinated 4-methylbutyrolactonefluorination reaction, as a crude compound as obtained (e.g., not further purified), is transferred into the mentioned another (second) microreactor, to be further reacted in a second reaction step by fluorination reaction with SF₄ as the fluorination agent (and, if applicable, involving the lactone ring opening) to yield the compound PFP (perfluoropentane), to yield the final target compound PFP (perfluoropentane).

In a further variant of the present invention, the final target compound PFP (perfluoropentane) can also be prepared out of the (intermediate) compound perfluorinated 4-methylbutyrolactone, and described herein above in more detail. Preferably, the reaction can be performed in a continuous manner.

Microreactor Process:

The invention also may pertain to a process for manufacturing of perfluoropentane (PFP), and/or of the compound perfluorinated 4-methylbutyrolactone, which is a suitable intermediate in the manufacture of perfluoropentane (PFP), wherein the process is a continuous process, preferably wherein the continuous process is carried out in a microreactor.

The invention may employ more than a single microreactor, i.e., the invention may employ two, three, four, five or more microreactors, for either extending the capacity or residence time, for example, to up to ten microreactors in parallel or four microreactors in series. If more than a single microreactor is employed, then the plurality of microreactors can be arranged either sequentially or in parallel, and if three or more microreactors are employed, these may be arranged sequentially, in parallel or both.

The invention is also very advantageous, in to embodiments wherein the process for manufacturing of the compound PFP (perfluoropentane), and/or of perfluorinated 4-methylbutyrolactone compound, i.e., the precursor or intermediate compound of PFP (perfluoropentane), according to the invention optionally is performed in a continuous flow reactor system, or preferably in a microreactor system.

In an preferred embodiment the invention relates to a process for manufacturing of the compound PFP (perfluoropentane), and/or of perfluorinated 4-methylbutyrolactone compound, i.e., the precursor or intermediate compound of PFP (perfluoropentane), wherein in at least one reaction step of direct fluorination reaction with F₂-gas as the fluorination agent, and/or the fluorination reaction with SF₄ as the fluorination agent (and, if applicable, involving the lactone ring opening) is carried out as a continuous processes, wherein the continuous process is performed in at least one continuous flow reactor with upper lateral dimensions of about ≤5 mm, or of about ≤4 mm, preferably in at least one microreactor.

In another preferred embodiment the invention relates to such a process of preparing a compound according to the invention, wherein at least one of the said continuous flow reactors, preferably at least one of the microreactors, independently is a SiC-continuous flow reactor, preferably independently is a SiC-microreactor.

The Continuous Flow Reactors and Microreactors:

In addition to the above, according to one aspect of the invention, also a plant engineering invention is provided, as used in the process invention and described herein, pertaining to the optional, and in some embodiments of the process invention, the process even preferred implementation in microreactors.

As to the term “microreactor”: A “microreactor” or “microstructured reactor” or “microchannel reactor”, in one embodiment of the invention, is a device in which chemical reactions take place in a confinement with typical lateral dimensions of about ≤1 mm; an example of a typical form of such confinement are microchannels. Generally, in the context of the invention, the term “microreactor”: A “microreactor” or “microstructured reactor” or “microchannel reactor”, denotes a device in which chemical reactions take place in a confinement with typical lateral dimensions of about ≤5 mm.

Microreactors are studied in the field of micro process engineering, together with other devices (such as micro heat exchangers) in which physical processes occur. The microreactor is usually a continuous flow reactor (contrast with/to a batch reactor). Microreactors offer many advantages over conventional scale reactors, including vast improvements in energy efficiency, reaction speed and yield, safety, reliability, scalability, on-site/on-demand production, and a much finer degree of process control.

Microreactors are used in “flow chemistry” to perform chemical reactions. In flow chemistry, wherein often microreactors are used, a chemical reaction is run in a continuously flowing stream rather than in batch production. Batch production is a technique used in manufacturing, in which the object in question is created stage by stage over a series of workstations, and different batches of products are made. Together with job production (one-off production) and mass production (flow production or continuous production) it is one of the three main production methods. In contrast, in flow chemistry the chemical reaction is run in a continuously flowing stream, wherein pumps move fluid into a tube, and where tubes join one another, the fluids contact one another. If these fluids are reactive, a reaction takes place. Flow chemistry is a well-established technique for use at a large scale when manufacturing large quantities of a given material. However, the term has only been coined recently for its application on a laboratory scale.

Continuous flow reactors, e.g. such as used as microreactor, are typically tube like and manufactured from non-reactive materials, such known in the prior art and depending on the specific purpose and nature of possibly aggressive agents and/or reactants. Mixing methods include diffusion alone, e.g. if the diameter of the reactor is narrow, e.g. <1 mm, such as in microreactors, and static mixers. Continuous flow reactors allow good control over reaction conditions including heat transfer, time and mixing. The residence time of the reagents in the reactor, i.e. the amount of time that the reaction is heated or cooled, is calculated from the volume of the reactor and the flow rate through it: Residence time=Reactor Volume/Flow Rate. Therefore, to achieve a longer residence time, reagents can be pumped more slowly, just a larger volume reactor can be used and/or even several microreactors can be placed in series, optionally just having some cylinders in between for increasing residence time if necessary for completion of reaction steps. In this later case, cyclones after each microreactor help to let escape some low boiling substances, e.g., any formed PFVME together with (potentially present) inert gas and so far to positively influence the reaction performance. Production rates can vary from milliliters per minute to liters per hour.

Some examples of flow reactors are spinning disk reactors (Colin Ramshaw); spinning tube reactors; multi-cell flow reactors; oscillatory flow reactors; microreactors; hex reactors; and aspirator reactors. In an aspirator reactor a pump propels one reagent, which causes a reactant to be sucked in. Also to be mentioned are plug flow reactors and tubular flow reactors.

In the present invention, in one embodiment it is particularly preferred to employ a microreactor.

In the use and processes according to the invention in a preferred embodiment the invention is using a microreactor. But it is to be noted in a more general embodiment of the invention, apart from the said preferred embodiment of the invention that is using a microreactor, any other, e.g. preferentially pipe-like, continuous flow reactor with upper lateral dimensions of up to about 1 cm, and as defined herein, can be employed. Thus, such a continuous flow reactor preferably with upper lateral dimensions of up to about ≤5 mm, or of about ≤4 mm, refers to a preferred embodiment of the invention, e.g. preferably to a microreactor. Continuously operated series of STRs is another option, but less preferred than using a microreactor.

In the before said embodiments of the invention, the minimal lateral dimensions of the, e.g. preferentially pipe-like, continuous flow reactor can be about >5 mm; but is usually not exceeding about 1 cm. Thus, the lateral dimensions of the, e.g. preferentially pipe-like, continuous flow reactor can be in the range of from about >5 mm up to about 1 cm, and can be of any value therein between. For example, the lateral dimensions of the, e.g. preferentially pipe-like, continuous flow reactor can be about 5.1 mm, about 5.5 mm, about 6 mm, about 6.5 mm, about 7 mm, about 7.5 mm, about 8 mm, about 8.5 mm, about 9 mm, about 9.5 mm, and about 10 mm, or can be can be of any value intermediate between the said values.

In the before said embodiments of the invention using a microreactor preferentially the minimal lateral dimensions of the microreactor can be at least about 0.25 mm, and preferably at least about 0.5 mm; but the maximum lateral dimensions of the microreactor does not exceed about ≤5 mm. Thus, the lateral dimensions of the, e.g. preferential microreactor can be in the range of from about 0.25 mm up to about ≤5 mm, and preferably from about 0.5 mm up to about ≤5 mm, and can be of any value therein between. For example, the lateral dimensions of the preferential microreactor can be about 0.25 mm, about 0.3 mm, about 0.35 mm, about 0.4 mm, about 0.45 mm, and about 5 mm, or can be can be of any value intermediate between the said values.

As stated here before in the embodiments of the invention in its broadest meaning is employing, preferentially pipe-like, continuous flow reactor with upper lateral dimensions of up to about 1 cm. Such continuous flow reactor, for example is a plug flow reactor (PFR).

The plug flow reactor (PFR), sometimes called continuous tubular reactor, CTR, or piston flow reactors, is a reactor used to perform and describe chemical reactions in continuous, flowing systems of cylindrical geometry. The PFR reactor model is used to predict the behavior of chemical reactors of such design, so that key reactor variables, such as the dimensions of the reactor, can be estimated.

Fluid going through a PFR may be modeled as flowing through the reactor as a series of infinitely thin coherent “plugs”, each with a uniform composition, traveling in the axial direction of the reactor, with each plug having a different composition from the ones before and after it. The key assumption is that as a plug flows through a PFR, the fluid is perfectly mixed in the radial direction (i.e. in the lateral direction) but not in the axial direction (forwards or backwards).

Accordingly, the terms used herein to define the reactor type used in the context of the invention such like “continuous flow reactor”, “plug flow reactor”, “tubular reactor”, “continuous flow reactor system”, “plug flow reactor system”, “tubular reactor system”, “continuous flow system”, “plug flow system”, “tubular system” are synonymous to each other and interchangeably by each other.

The reactor or system may be arranged as a multitude of tubes, which may be, for example, linear, looped, meandering, circled, coiled, or combinations thereof. If coiled, for example, then the reactor or system is also called “coiled reactor” or “coiled system”.

In the radial direction, i.e. in the lateral direction, such reactor or system may have an inner diameter or an inner cross-section dimension (i.e. radial dimension or lateral dimension, respectively) of up to about 1 cm. Thus, in an embodiment the lateral dimension of the reactor or system may be in the range of from about 0.25 mm up to about 1 cm, preferably of from about 0.5 mm up to about 1 cm, and more preferably of from about 1 mm up to about 1 cm.

In further embodiments the lateral dimension of the reactor or system may be in the range of from about >5 mm to about 1 cm, or of from about 5.1 mm to about 1 cm.

If the lateral dimension at maximum of up to about ≤5 mm, or of up to about ≤4 mm, then the reactor is called “microreactor”. Thus, in still further microreactor embodiments the lateral dimension of the reactor or system may be in the range of from about 0.25 mm up to about ≤5 mm, preferably of from about 0.5 mm up to about ≤5 mm, and more preferably of from about 1 mm up to about ≤5 mm; or the lateral dimension of the reactor or system may be in the range of from about 0.25 mm up to about ≤4 mm, preferably of from about 0.5 mm up to about ≤4 mm, and more preferably of from about 1 mm up to about ≤4 mm.

In an alternative embodiment of the invention, it is also optionally desired to employ another continuous flow reactor than a microreactor, preferably if, for example, the (halogenation promoting, e.g. the halogenation or preferably the halogenation) catalyst composition used in the halogenation or fluorination tends to get viscous during reaction or is viscous already as a said catalyst as such. In such case, a continuous flow reactor, i.e. a device in which chemical reactions take place in a confinement with lower lateral dimensions of greater than that indicated above for a microreactor, i.e. of greater than about 1 mm, but wherein the upper lateral dimensions are about ≤4 mm. Accordingly, in this alternative embodiment of the invention, employing a continuous flow reactor, the term “continuous flow reactor” preferably denotes a device in which chemical reactions take place in a confinement with typical lateral dimensions of from about ≥1 mm up to about ≤4 mm. In such an embodiment of the invention it is particularly preferred to employ as a continuous flow reactor a plug flow reactor and/or a tubular flow reactor, with the said lateral dimensions. Also, in such an embodiment of the invention, as compared to the embodiment employing a microreactor, it is particularly preferred to employ higher flow rates in the continuous flow reactor, preferably in the plug flow reactor and/or a tubular flow reactor, with the said lateral dimensions. For example, such higher flow rates, are up to about 2 times higher, up to about 3 times higher, up to about 4 times higher, up to about 5 times higher, up to about 6 times higher, up to about 7 times higher, or any intermediate flow rate of from about ≥1 up to about ≤7 times higher, of from about ≥1 up to about ≤6 times higher, of from about ≥1 up to about ≤5 times higher, of from about ≥1 up to about ≤4 times higher, of from about ≥1 up to about ≤3 times higher, or of from about ≥1 up to about ≤2 times higher, each as compared to the typical flow rates indicated herein for a microreactor. Preferably, the said continuous flow reactor, more preferably the the plug flow reactor and/or a tubular flow reactor, employed in this embodiment of the invention is configured with the construction materials as defined herein for the microreactors. For example, such construction materials are silicon carbide (SiC) and/or are alloys such as a highly corrosion resistant nickel-chromium-molybdenum-tungsten alloy, e.g. Hastelloy®, as described herein for the microreactors.

A very particular advantage of the present invention employing a microreactor, or a continuous flow reactor with the before said lateral dimensions, the number of separating steps can be reduced and simplified, and may be devoid of time and energy consuming, e.g. intermediate, distillation steps. Especially, it is a particular advantage of the present invention employing a microreactor, or a continuous flow reactor with the before said lateral dimensions, that for separating simply phase separation methods can be employed, and the non-consumed reaction components may be recycled into the process, or otherwise be used as a product itself, as applicable or desired.

In addition to the preferred embodiments of the present invention using a microreactor according to the invention, in addition or alternatively to using a microreactor, it is also possible to employ a plug flow reactor or a tubular flow reactor, respectively.

Plug flow reactor or tubular flow reactor, respectively, and their operation conditions, are well known to those skilled in the field.

Although the use of a continuous flow reactor with upper lateral dimensions of about ≤5 mm, or of about ≤4 mm, respectively, and in particular of a microreactor, is particularly preferred in the present invention, depending on the circumstances, it could be imagined that somebody dispenses with an microreactor, then of course with yield losses and higher residence time, higher temperature, and instead takes a plug flow reactor or turbulent flow reactor, respectively. However, this could have a potential advantage, taking note of the mentioned possibly disadvantageous yield losses, namely the advantage that the probability of possible blockages (tar particle formation by non-ideal driving style) could be reduced because the diameters of the tubes or channels of a plug flow reactor are greater than those of a microreactor.

The possibly allegeable disadvantage of this variant using a plug flow reactor or a tubular flow reactor, however, may also be seen only as subjective point of view, but on the other hand under certain process constraints in a region or at a production facility may still be appropriate, and loss of yields be considered of less importance or even being acceptable in view of other advantages or avoidance of constraints.

In the following, the invention is more particularly described in the context of using a microreactor. Preferentially, a microreactor used according to the invention is a ceramic continuous flow reactor, more preferably an SiC (silicon carbide) continuous flow reactor, and can be used for material production at a multi-to scale. Within integrated heat exchangers and SiC materials of construction, it gives optimal control of challenging flow chemistry application. The compact, modular construction of the flow production reactor enables, advantageously for: long term flexibility towards different process types; access to a range of production volumes (5 to 400 l/h); intensified chemical production where space is limited; unrivalled chemical compatibility and thermal control.

Ceramic (SiC) microreactors, are e.g. advantageously diffusion bonded 3M SiC reactors, especially braze and metal free, provide for excellent heat and mass transfer, superior chemical compatibility, of FDA certified materials of construction, or of other drug regulatory authority (e.g. EMA) certified materials of construction. Silicon carbide (SiC), also known as carborundum, is a containing silicon and carbon, and is well known to those skilled in the art. For example, synthetic SiC powder is been mass-produced and processed for many technical applications.

For example, in the embodiments of the invention the objects are achieved by a method in which at least one reaction step takes place in a microreactor. Particularly, in preferred embodiments of the invention the objects are achieved by a method in which at least one reaction step takes place in a microreactor that is comprising or is made of SiC (“SiC-microreactor”), or in a microreactor that is comprising or is made of an alloy, e.g. such as Hastelloy C, as it is each defined herein after in more detail.

Preferred Hastelloy C4 nickel alloys are already described further above. See, for example, Table 1.

Thus, without being limited to, for example, in an embodiment of the invention the microreactor suitable for, preferably for industrial, production an “SiC-microreactor” that is comprising or is made of SiC (silicon carbide; e.g. SiC as offered by Dow Corning as Type G1SiC or by Chemtrix MR555 Plantrix), e.g. providing a production capacity of from about 5 up to about 400 kg per hour; or without being limited to, for example, in another embodiment of the invention the microreactor suitable for industrial production is comprising or is made of Hastelloy C, as offered by Ehrfeld. Such microreactors are particularly suitable for the, preferably industrial, production of fluorinated products according to the invention.

In order to meet both the mechanical and chemical demands placed on production scale flow reactors, Plantrix modules are fabricated from 3M™ SiC (Grade C). Produced using the patented 3M (EP 1 637 271 Bi and foreign patents) diffusion bonding technology, the resulting monolithic reactors are hermetically sealed and are free from welding lines/joints and brazing agents. More technical information on the Chemtrix MR555 Plantrix can be found in the brochure “CHEMTRIX—Scalable Flow Chemistry—Technical Information Plantrix® MR555 Series, published by Chemtrix BV in 2017, which technical information is incorporated herein by reference in its entirety.

Apart from the before said example, in other embodiments of the invention, in general SiC from other manufactures, and as known to the skilled person, of course can be employed in the present invention.

Accordingly, in the present invention as microreactor also the Protrix® of by Chemtrix can be used. Protrix® is a modular, continuous flow reactor fabricated from 3M® silicon carbide, offering superior chemical resistance and heat transfer. In order to meet both the mechanical and chemical demands placed on flow reactors, Protrix® modules are fabricated from 3M® SiC (Grade C). Produced using the patented 3M (EP 1 637 271 Bi and foreign patents) diffusion bonding technology, the resulting monolithic reactors are hermetically sealed and are free from welding lines/joints and brazing agents. This fabrication technique is a production method that gives solid SiC reactors (thermal expansion coefficient=4.1×10⁻⁶K⁻¹).

Designed for flow rates ranging from 0.2 to 20 ml/min and pressures up to 25 bar, Protrix® allows the user to develop continuous flow processes at the lab-scale, later transitioning to Plantrix® MR555 (x340 scale factor) for material production. The Protrix® reactor is a unique flow reactor with the following advantages: diffusion bonded 3M® SiC modules with integrated heat exchangers that offer unrivaled thermal control and superior chemical resistance; safe employment of extreme reaction conditions on a g scale in a standard fume hood; efficient, flexible production in terms of number of reagent inputs, capacity or reaction time. The general specifications for the Protrix® flow reactors are summarized as follows; possible reaction types are, e.g. A+B→P1+Q (or C)→P, wherein the terms “A”, “B” and “C” represent educts, “P” and “P1” products, and “Q” quencher; throughput (ml/min) of from about 0.2 up to about 20; channel dimensions (mm) of1×1 (pre-heat and mixer zone), 1.4×1.4 (residence channel); reagent feeds of 1 to 3; module dimensions (width×height) (mm) of 110×260; frame dimensions (width×height×length) (mm) approximately 400×300×250; number of modules/frame is one (minimum) up to four (max). More technical information on the Chemtrix Protrix® reactor can be found in the brochure “CHEMTRIX—Scalable Flow Chemistry—Technical Information Protrix®, published by Chemtrix BV in 2017, which technical information is incorporated herein by reference in its entirety.

The Dow Corning as Type G1SiC microreactor, which is scalable for industrial production, and as well suitable for process development and small production can be characterized in terms of dimensions as follows: typical reactor size (length×width×height) of 88 cm×38 cm×72 cm; typical fluidic module size of 188 mm×162 mm. The features of the Dow Corning as Type G1SiC microreactor can be summarized as follows: outstanding mixing and heat exchange: patented HEART design; small internal volume; high residence time; highly flexible and multipurpose; high chemical durability which makes it suitable for high pH compounds and especially hydrofluoric acid; hybrid glass/SiC solution for construction material; seamless scale-up with other advanced-flow reactors. Typical specifications of the Dow Corning as Type G1SiC microreactor are as follows: flow rate of from about 30 ml/min up to about 200 ml/min; operating temperature in the range of from about −60° C. up to about 200° C., operating pressure up to about 18 barg (“barg” is a unit of gauge pressure, i.e. pressure in bars above ambient or atmospheric pressure); materials used are silicon carbide, PFA (perfluoroalkoxy alkanes), perfluoroelastomer; fluidic module of 10 ml internal volume; options: regulatory authority certifications, e.g. FDA or EMA, respectively. The reactor configuration of Dow Corning as Type G1SiC microreactor is characterized as multipurpose and configuration can be customized. Injection points may be added anywhere on the said reactor.

Hastelloy® C is an alloy represented by the formula NiCr21Mol4W, alternatively also known as “alloy 22” or “Hastelloy® C-22. The said alloy is well known as a highly corrosion resistant nickel-chromium-molybdenum-tungsten alloy and has excellent resistance to oxidizing reducing and mixed acids. The said alloy is used in flue gas desulphurization plants, in the chemical industry, environmental protection systems, waste incineration plants, sewage plants. Apart from the before said example, in other embodiments of the invention, in general nickel-chromium-molybdenum-tungsten alloy from other manufactures, and as known to the skilled person, of course can be employed in the present invention. A typical chemical composition (all in weight-%) of such nickel-chromium-molybdenum-tungsten alloy is, each percentage based on the total alloy composition as 100%: Ni (nickel) as the main component (balance) of at least about 51.0%, e.g. in a range of from about 51.0% to about 63.0%; Cr (chromium) in a range of from about 20.0 to about 22.5%, Mo (molybdenum) in a range of from about 12.5 to about 14.5%, W (tungsten or wolfram, respectively) in a range of from about 2.5 to about 3.5%; and Fe (iron) in an amount of up to about 6.0%, e.g. in a range of from about 1.0% to about 6.0%, preferably in a range of from about 1.5% to about 6.0%, more preferably in a range of from about 2.0% to about 6.0%. Optionally, the percentage based on the total alloy composition as 100%, Co (cobalt) can be present in the alloy in an amount of up to about 2.5%, e.g. in a range of from about 0.1% to about 2.5%. Optionally, the percentage based on the total alloy composition as 100%, V (vanadium) can be present in the alloy in an amount of up to about 0.35%, e.g. in a range of from about 0.1% to about 0,35%. Also, the percentage based on the total alloy composition as 100%, optionally low amounts (i.e. ≤0.1%) of other element traces, e.g. independently of C (carbon), Si (silicon), Mn (manganese), P (phosphor), and/or S (sulfur). In such case of low amounts (i.e. ≤0.1%) of other elements, the said elements e.g. of C (carbon), Si (silicon), Mn (manganese), P (phosphor), and/or S (sulfur), the percentage based on the total alloy composition as 100%, each independently can be present in an amount of up to about 0.1%, e.g. each independently in a range of from about 0.01 to about 0.1%, preferably each independently in an amount of up to about 0.08%, e.g. each independently in a range of from about 0.01 to about 0.08%. For example, said elements e.g. of C (carbon), Si (silicon), Mn (manganese), P (phosphor), and/or S (sulfur), the percentage based on the total alloy composition as 100%, each independently can be present in an amount of, each value as an about value: C≤0.01%, Si≤0.08%, Mn≤0.05%, P≤0.015%, S≤0.02%. Normally, no traceable amounts of any of the following elements are found in the alloy compositions indicated above: Nb (niobium), Ti (titanium), Al (aluminum), Cu (copper), N (nitrogen), and Ce (cerium).

Hastelloy® C-276 alloy was the first wrought, nickel-chromium-molybdenum material to alleviate concerns over welding (by virtue of extremely low carbon and silicon contents). As such, it was widely accepted in the chemical process and associated industries, and now has a 50-year-old track record of proven performance in a vast number of corrosive chemicals. Like other nickel alloys, it is ductile, easy to form and weld, and possesses exceptional resistance to stress corrosion cracking in chloride-bearing solutions (a form of degradation to which the austenitic stainless steels are prone). With its high chromium and molybdenum contents, it is able to withstand both oxidizing and nonoxidizing acids, and exhibits outstanding resistance to pitting and crevice attack in the presence of chlorides and other halides. The nominal composition in weight-% is, based on the total composition as 100%: Ni (nickel) 57% (balance); Co (cobalt) 2.5% (max); Cr (chromium) 16%; Mo (molybdenum) 16%; Fe (iron) 5%; W (tungsten or wolfram, respectively) 4%; further components in lower amounts can be Mn (manganese) up to 1% (max); V (vanadium) up to 0.35% (max); Si (silicon) up to 0.08% (max); C (carbon) 0.01 (max); Cu (copper) up to 0.5% (max).

In another embodiments of the invention, without being limited to, for example, the microreactor suitable for the said production, preferably for the said industrial production, is an SiC-microreactor that is comprising or is made only of SiC as the construction material (silicon carbide; e.g. SiC as offered by Dow Corning as Type G1SiC or by Chemtrix MR555 Plantrix), e.g. providing a production capacity of from about 5 up to about 400 kg per hour.

It is of course possible according to the invention to use one or more microreactors, preferably one or more SiC-microreactors, in the production, preferably in the industrial production, of the fluorinated products according to the invention. If more than one microreactor, preferably more than one SiC-microreactor, are used in the production, preferably in the industrial production, of the fluorinated products according to the invention, then these microreactors, preferably these SiC-microreactors, can be used in parallel and/or subsequent arrangements. For example, two, three, four, or more microreactors, preferably two, three, four, or more SiC-microreactors, can be used in parallel and/or subsequent arrangements.

For laboratory search, e.g. on applicable reaction and/or upscaling conditions, without being limited to, for example, as a microreactor the reactor type Plantrix of the company Chemtrix is suitable. Sometimes, if gaskets of a microreactor are made out of other material than HDPTFE, leakage might occur quite soon after short time of operation because of some swelling, so HDPTFE gaskets secure long operating time of microreactor and involved other equipment parts like settler and distillation columns.

For example, an industrial flow reactor (“IFR”, e.g. Plantrix® MR555) comprises of SiC modules (e.g. 3M® SiC) housed within a (non-wetted) stainless steel frame, through which connection of feed lines and service media are made using standard Swagelok fittings. The process fluids are heated or cooled within the modules using integrated heat exchangers, when used in conjunction with a service medium (thermal fluid or steam), and reacted in zig-zag or double zig-zag, meso-channel structures that are designed to give plug flow and have a high heat exchange capacity. A basic IFR (e.g. Plantrix® MR555) system comprises of one SiC module (e.g. 3M® SiC), a mixer (“MRX”) that affords access to A+B→P type reactions. Increasing the number of modules leads to increased reaction times and/or system productivity. The addition of a quench Q/C module extends reaction types to A+B→P1+Q (or C)→P and a blanking plate gives two temperature zones. Herein the terms “A”, “B” and “C” represent educts, “P” and “P1” products, and “Q” quencher.

Typical dimensions of an industrial flow reactor (“IFR”, e.g. Plantrix® MR555) are, for example: channel dimensions in (mm) of 4×4 (“MRX”, mixer) and 5×5 (MRHI/MRH-II; “MRH” denotes residence module); module dimensions (width×height) of 200 mm×555 mm; frame dimensions (width×height) of 322 mm×811 mm. A typical throughput of an industrial flow reactor (“IFR”, e.g. Plantrix® MR555) is, for example, in the range of from about 50 l/h to about 400 l/h. in addition, depending on fluid properties and process conditions used, the throughput of an industrial flow reactor (“IFR”, e.g. Plantrix® MR555), for example, can also be >400 l/h. The residence modules can be placed in series in order to deliver the required reaction volume or productivity. The number of modules that can be placed in series depends on fluid properties and targeted flow rate.

Typical operating or process conditions of an industrial flow reactor (“IFR”, e.g. Plantrix® MR555) are, for example: temperature range of from about −30° C. to about 200° C.; temperature difference (service−process)<70° C.; reagent feeds of 1 to 3; maximum operating pressure (service fluid) of about 5 bar at a temperature of about 200° C.; maximum operating pressure (process fluid) of about 25 bar at a temperature of about ≤200° C.

### EXAMPLES

The following examples are intended to further illustrate the invention without limiting its scope.

### Example 1

Both steps done in an autoclave:

A 250 ml Roth autoclave with HDPTFE-inliner (made by company Berghof Fluoroplastics), magnetic stirrer, deep pipe and outlet with pressure valve over the gas phase (with an efficient scrubber after the valve) was filled with 100 g (1.0 mol) 4-methylbutyrolactone (+/−) and put into an ice bath. 20% F₂ (80% N₂) was fed out of a gas cylinder over the deep pipe at room temperature into the autoclave, the pressure valve was adjusted to 5 bar while N₂ and formed HF were leaving the autoclave together. The F₂-dosage was done in that manner that the temperature in the autoclave did NOT exceed 40° C. After 4 h, no exothermic activity could be observed any more. Based on the 20% F₂, 380 g (10.0 mol) F₂ were consumed until the exothermic activity has significantly dropped down. Stirring was continued for 1 more h at 5 bar pressure. The autoclave was not degassed to make sure that HF has stayed as solvent for the next step in the solution. The pressure valve now was adjusted to a pressure of 8 bar and the autoclave was heated to 50° C. 162.75 g (1.5 mol) SF₄ were now fed out of another gas cylinder into the autoclave, some formed SOF₂ and excess SF₄ were allowed to leave while the reaction took place. After having finished the SF₄-dosage, stirring was continued for 3 h at 50° C. before the autoclave was let cooled down to 0° C. Now a N₂-stream was fed over the deep pipe through the solution to get rid of HF and SOF₂. Afterwards, the entire autoclave content was carefully poured into ice water, the organic phase was dried over Na₂SO₄ before PFP was distilled off at 30° C. transition temperature at atmospheric pressure to obtain 132.5 g PFP with a purity of 98.2% (GC).

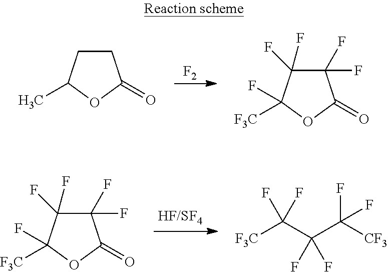

### Example 2

Both steps done in a Batch synthesis and in a loop reactor.

Apparatus: A column with a length of 30 cm with PTFE fillings and a diameter of 5 cm was used according to the drawing below. The liquid reservoir had a volume of 2 l. The pump was a centrifugal pump from company Schmitt. A pressure valve on top of the tower was installed to regulate the pressure, a cooling trap was installed after the pressure valve which was in use for the 2^{nd} step only to collect some PFP leaving with the gas stream.

See FIG. 1 for apparatus and reaction.

The reservoir was filled with 1 kg (9.99 mol) 4-Methylbutyrolactone (+/−) and the pump was started (flow ˜1500 l/h). 10% F₂-gas (in N₂) was fed over a Bronkhorst mass flow meter into the tower so that the reaction temperature was kept at 30° C. while the pressure on the tower was kept at 2 bar abs. by the pressure valve. After 1 h 3.04 kg (80.0 mol) F₂ were fed into the system while the inert N₂ together with HF left the apparatus over the pressure valve over the top into an efficient scrubber. After 10 min of further looping without any dosage, SF₄ feed out of another cylinder was started also using a Bronkhorst mass flow meter. 2.26 kg (20.8 mol) SF₄ were fed over 2 h into the loop at bottom of the tower so while the tower was kept at 30° C. After 10 min of further looping, the pump was stopped and 50 g (0.57 mol NEt₃) was added to neutralize some remaining HF (all SOF₂ and excess SF₄ had left the system already), a 2^{nd} phase was formed containing the amine/HF adduct. After Phase separation, PFP in the lower phase was distilled over a Vigreux column at 29.9° C. transition temperature at atmospheric pressure to obtain 2.45 kg PFP (85% yield) with a purity of 99.9% (GC).

### Example 3

Continuous preparation of PFP in microreactor system

See FIG. 2 for apparatus and reaction.

One 27 ml Microreactor from Chemtrix made out of SiC was used for the 1^{st} step, 2 in series connected 27 ml microreactors were used for 2^{nd} step. All 3 microreactors were operated at 30° C., the pressure after 1^{st} microreactor was adjusted to 5 bar by using a pressure valve installed at HF/inertgas outlet at the cyclone, which is not shown in the drawing. Pressure after 2^{nd} microreactor is adjusted to 2 bar abs. by a pressure valve installed at SOF₂ outlet at another cyclone, which is also not shown in the drawing. The raw material reservoir contains a double wall jacket and is cooled to 0° C. 100 g (1.0 mol) 4-methylbutyrolactone was fed together with 323 g (8.5 mol) F₂ directly from a fluorine cell additional diluted with 10% N₂ over a Bronkhorst mass flow meter and over 1 h into the 1^{st} microreactor, a very strong exothermicity was observed so that a cooling machine (−20° C.) had to be used to keep the reaction temperature in the microreactor at 30° C. Around half of the formed HF was purged over the Cyclone (HF left together with the N₂) after the 1^{st} microreactor before the reaction mixture entered the 2^{nd} microreactor together with 227.0 g (2.1 mol) SF₄ fed out of another cylinder over a Bronkhorst mass flow meter over this 1 h reaction time. Before starting the feed, the raw material reservoir was preloaded already with 50 g NEt₃ to neutralize some potential HF amounts which even after the purge after the 2×27 ml microreactors (used for the SF₄) step still is present in the product mixture. Alternatively and as drawn in the reaction scheme, NEt₃ could be fed continuously corresponding to the remaining HF amount after the SF₄-step. After around 18 min after reaction start, in the raw product reservoir, the formation of a 2^{nd} (lower) phase containing mainly PFP could be detected. After phase separation and additional distillation over a 20 cm Vigreux column (30.0° C. transition temperature, 1 bar absol.), PFP with a purity of 99.99% (GC) in 94% isolated yield could be obtained.

### Example 4

PFP Synthesis Out of Hexafluoro Acetylacetone (HFAA) in Microreactors

Same equipment as in example 3 was used except that the 2×27 ml reactors were installed first and a new Teflon coated tank was used for hexafluoro acetylacetone (HFAA) raw material storage.

See FIG. 3 for apparatus and reaction.

Commercial available hexafluoro acetylacetone (HFAA) dissolved in 20 ml water free HF, per hour 280.0 g (1.35 mol) HFAA, was fed together with 302.5 g (2.8 mol) SF₄ into the 1^{st} microreactor part (2×27 ml) constantly over 1 h. The reaction temperature was kept at 50° C. SOF₂ together with some HF and traces SF₄ left over the cyclone installed after the 1^{st} part into an efficient scrubber. Pressure was adjusted to 5 bar abs. by a pressure valve. In parallel during this hour, feed of 106.4 (2.8 mol) F₂ (diluted with 10% N₂) was fed before 2^{nd} microreactor which was adjusted to 30° C. and 3 bar abs. NEt₃ was fed continuously into the reaction stream to neutralize the HF (a cooler is installed at this position which is not drawn in the scheme as this neutralization is exothermic). This yielded 97% PFP as 2^{nd} lower phase in the raw material reservoir which after final distillation, gave a yield of 95% (369 g) PFP with a purity of 99.99%. As alternative work up, especially for larger scale production, all material after the 2^{nd} microreactor step (without NEt₃ treatment) can be collected in a cooled trap cooled to −5° C. and PFP/HF mixture and can be fractioned at 5 bar abs. in a stainless steel column to separate HF (which can be re-used) and the product PFP.

### Example 5

PFP synthesis out of trifluoro acetylacetone in an autoclave without solvent.

A 250 ml Roth autoclave with HDPTFE-inliner (made by company Berghof Fluoroplastics), magnetic stirrer, deep pipe and outlet with pressure valve over the gas phase (with an efficient scrubber after the valve) was filled with 100 g (0.65 mol) 1,1,1-Trifluoro-2,4-pentanedione (TFAA) and put into an ice bath. 162.75 g (1.5 mol) SF₄ were now fed out of a gas cylinder into the autoclave and heated to 50° C. in an oil bath for 2 h, the pressure raised to 10 bar abs. After cooling down to room temperature, the pressure (SOF₂) was released over the gas phase outlet into an efficient scrubber to a pressure in the autoclave of 1 bar abs. The autoclave was put into an ice bath and now 20% F₂ (80% N₂) was fed out of a gas cylinder continuously over the deep pipe very slowly into the autoclave while the pressure valve was adjusted to 5 bar. The F₂-dosage was done in that manner that the temperature in the autoclave did NOT exceed 5° C. After a few minutes N₂ and formed HF were leaving the autoclave over the pressure valve into the efficient scrubber. After 3 h, exothermic activity formation started to slow down and after 4.5 h no heat formation could be observed any more. Based on the 20% F₂, 152 g (4.0 mol) F₂ were consumed until the exothermic activity has significantly dropped down (some F₂ has left together with N₂/HF into the scrubber). Stirring was continued for 1 more h at 5 bar abs. pressure. The autoclave content was slowly released into ice water over the deep pipe and adding some N₂-pressure at gas phase inlet. The organic phase was separated, neutralized and dried over Na₂SO₄ before PFP was distilled off at 30° C. transition temperature at atmospheric pressure to obtain 122 g PFP with a purity of 98.8% (GC) which corresponds to an isolated yield of 65%.

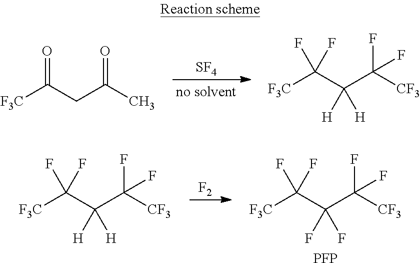

What is claimed is:

1. A process for the manufacture of the perfluorinated lactone compound having the formula (IV), which is perfluorinated 4-methylbutyrolactone, 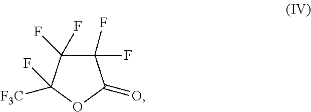 [SMILES: O=C1OC(F)(C(F)(F)F)C(F)(F)C1(F)F]
wherein the perfluorinated 4-methylbutyrolactone compound is manufactured starting from a starting material compound of formula (II), which is 4-methylbutyrolactone, 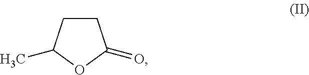
and
wherein the process is performed in a reactor or reactor system, resistant to elemental fluorine (F₂) and to hydrogen fluoride (HF), and the process comprises a direct fluorination reaction (i) with elemental fluorine (F₂) as gaseous fluorination agent, to yield the perfluorinated 4-methylbutyrolactone compound of formula (IV), which is perfluorinated 4-methylbutyrolactone.

2. A perfluorinated lactone compound having the formula (IV), which is perfluorinated 4-methylbutyrolactone,

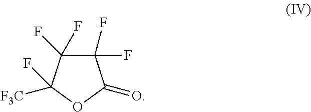 [SMILES: O=C1OC(F)(C(F)(F)F)C(F)(F)C1(F)F]

3. A process for the manufacture of the compound PFP (perfluoropentane) having the formula (I), 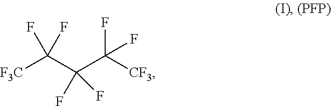 [SMILES: FC(F)(F)C(F)(F)C(F)(F)C(F)(F)C(F)(F)F]
wherein the compound PFP is manufactured starting from a perfluorinated lactone compound having the formula (IV), which is perfluorinated 4-methylbutyrolactone, 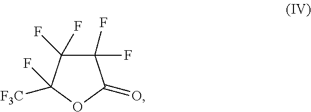 [SMILES: O=C1OC(F)(C(F)(F)F)C(F)(F)C1(F)F]
wherein the process is performed in a reactor or reactor system, resistant to elemental fluorine (F₂) and to hydrogen fluoride (HF), and the process comprises a fluorination reaction (ii) with a fluorination agent selected from the group consisting of SF₄, commercial fluorinating agents like DAST, Deoxo-Fluor, Xtal-Fluor (M/E), Fluorlead and PhenoFluor, preferably SF₄ as the fluorination agent, to yield the compound PFP (perfluoropentane) having the formula (I).

4. The process for the manufacture of the compound PFP (perfluoropentane) having the formula (I), according to claim 3, wherein the fluorination reaction (ii) is performed with SF₄ as the fluorination agent.

5. The process for the manufacture of the compound PFP (perfluoropentane) having the formula (I), according to claim 4, wherein the fluorination reaction (ii) is performed with SF₄ as the fluorination agent, and in HF (hydrogen fluoride) as solvent.

6. The process for the manufacture of the compound PFP (perfluoropentane) having the formula (I), according to claim 4, wherein the fluorination reaction (ii) is performed with SF₄ as the fluorination agent, and in HF (hydrogen fluoride) as solvent, and in the presence of a Lewis acid.

7. The process for the manufacture of the compound PFP (perfluoropentane) having the formula (I), according to claim 6, wherein the Lewis acid is selected from the group consisting of Lewis acids like TiClₙF₄₋ₙ, SnClnF₄₋ₙ, SbClₘF₅₋ₘ, wherein n denotes an integer of 0 to 4, and m denotes an integer of 0 to 5.

8. The process according to claim 4, wherein the direct fluorination reaction with F₂-gas as the fluorination agent, and/or the fluorination reaction with SF₄ as the fluorination agent and, if applicable, involving the lactone ring opening, is carried out in an autoclave, in a column reactor, a counter-current reactor system, in particular in a loop reactor system, or in a counter-current system, in a tube reactor system, a continuous flow reactor system, in a coil reactor system or in a microreactor system, preferably in a counter-current reactor system or in a microreactor.

9. The process according to claim 8, wherein the direct fluorination reaction with F₂-gas as the fluorination agent, and/or the fluorination reaction with SF₄ as the fluorination agent and, if applicable, involving the lactone ring opening, is carried out in a counter-current reactor system which is a column reactor, preferably wherein the counter-current reactor system is a column reactor, preferably a packed bed tower, more preferred a packed bed tower in the form of a gas scrubber system.

10. The process according to claim 9, wherein for the direct fluorination reaction with F₂-gas as the fluorination agent, and/or the for the fluorination reaction with SF₄ as the fluorination agent and, if applicable, involving the lactone ring opening, the (closed) column reactor is equipped with at least one of the following:
(i) at least one heat exchanger, at least one liquid reservoir, with inlet and outlet for, and containing the liquid reaction medium;
(ii) a pump for pumping and circulating the liquid reaction medium;
(iii) one or more jets, preferably wherein the one or more (nozzle) jets are placed at the top of the column reactor, for spraying the circulating reaction medium into the column reactor;
(iv) one or more feeding inlets for introducing the fluorination gas comprising or consisting of elemental fluorine (F2) and/or SF4 as the fluorination agent into the (closed) column reactor;
(v) optionally one or more sieves, preferably two sieves, preferably the one or more sieves placed at the bottom of the column reactor;
(vi) and at least one gas outlet equipped with a pressure valve, and at least one outlet for withdrawing the fluorinated compound from the column reactor.

11. The process according to claim 9, wherein column reactor is a packed bed tower reactor, preferably a packed bed tower reactor is packed with fillers resistant to the reactants and especially resistant to elemental fluorine (F₂) and to hydrogen fluoride (HF) such as, e.g., with Raschig fillers, E-TFE fillers, and/or HF-resistant metal fillers, e.g., Hastelloy metal fillers, and/or HDPTFE-fillers or PTFE-fillers, more preferably wherein the packed bed tower reactor is a gas scrubber system which is packed with any of the before mentioned HF-resistant Hastelloy metal fillers and/or HDPTFE-fillers or PTFE-fillers, and preferably with HDPTFE-fillers or PTFE-fillers.

12. The process according to claim 8, wherein the direct fluorination reaction with F₂-gas as the fluorination agent, and/or the fluorination reaction with SF₄ as the fluorination agent and, if applicable, involving the lactone ring opening, is carried out in at least one step in a continuous flow reactor with upper lateral dimensions of about ≤5 mm, or of about ≤4 mm, more preferably in at least one step in a microreactor;
still more preferably wherein the direct fluorination reaction with F₂-gas as the fluorination agent, and/or the fluorination reaction with SF₄ as the fluorination agent and, if applicable, involving the lactone ring opening, is carried out in at least in one step as a continuous processes, wherein the continuous process is performed in at least one continuous flow reactor with upper lateral dimensions of about ≤5 mm, or of about ≤4 mm;
even more preferably wherein the direct fluorination reaction with F₂-gas as the fluorination agent, and/or the fluorination reaction with SF₄ as the fluorination agent and, if applicable, involving the lactone ring opening, is carried out in at least in one step as a continuous processes, wherein the continuous process is performed in at least one microreactor.

13. The process according to claim 12, characterized in that the direct fluorination reaction with F₂-gas as the fluorination agent, and/or the fluorination reaction with SF₄ as the fluorination agent and, if applicable, involving the lactone ring opening, is performed in a SiC-reactor.

14. The process according to claim 3, characterized in that prior to starting any of the process steps of the direct fluorination reaction with F₂-gas as the fluorination agent, and/or the fluorination reaction with SF₄ as the fluorination agent and, if applicable, involving the lactone ring opening, one or more of the reactors used, preferably each and any of the reactors used, are purged with an inert gas or a mixture of inert gases, preferably with He (helium) and/or N₂ (nitrogen) as the inert gas, more preferably with N₂ (nitrogen) as the inert gas.

15. The process according to claim 3, characterized in that prior to starting any of the process steps of the direct fluorination reaction with F₂-gas as the fluorination agent, and/or the fluorination reaction with SF₄ as the fluorination agent and, if applicable, involving the lactone ring opening, is performed in a nickel-reactor (Ni-reactor) or in a reactor with an inner surface with high nickel-content (Ni-content).

16. The process according to claim 3, for the manufacture of the compound PFP (perfluoropentane), and/or of the compound perfluorinated 4-methylbutyrolactone, i.e., the precursor or intermediate compound of PFP (perfluoropentane), characterized in that, independently, the product yielding from the direct fluorination reaction with F₂-gas as the fluorination agent, and/or from the fluorination reaction with SF₄ as the fluorination agent and, if applicable, involving the lactone ring opening, are subjected to distillation.
